(19) United States
(12) Patent Application Publication
(10) Pub. No.: US 2026/0326367 A1
(43) Pub. Date: Oct. 22, 2026

(54) PRINTED UNIVERSAL GRADIENT INTERFACES FOR BIOELECTRONIC DEVICES AND METHODS OF MAKING THE SAME

(71) Applicant: UNIVERSITY OF NOTRE DAME DU LAC, South Bend, IN (US)

(72) Inventor: Yanliang ZHANG, South Bend, IN (US)

(21) Appl. No.: 19/468,498

(22) Filed: Feb. 3, 2026

Related U.S. Application Data
(60) Provisional application No. 63/753,619, filed on Feb. 4, 2025.

(51) Int. Cl. B29C 64/112; A61B 5/00; A61B 5/0205; A61B 5/024; A61B 5/0255; B29C 64/393; C09D 11/38; C09D 11/52

(52) U.S. Cl. CPC B29C 64/112; A61B 5/02055; A61B 5/6824; A61B 5/6869; B29C 64/393; C09D 11/38; C09D 11/52; A61B 5/02444; A61B 5/0255; A61B 2562/164

(57) ABSTRACT

Described herein is a printed universal gradient interface (UGI) for stretchable and strain-insensitive bioelectronics. The UGI comprises a multi-polymer gradient extending between a first polymer end and a second polymer end with a graded weight ratio from 1:0 to 0:1. The UGI may connect a biological surface to an electrically conductive material, enabling improved strain insensitivity and electrical stability under high stretch ratios. Methods of making the UGI via aerosol jet printing techniques and bioelectronic devices incorporating the UGI with electrically conductive material are also described.

### CROSS-REFERENCE TO RELATED APPLICATIONS

This application claims priority to U.S. Provisional Patent Application No. 63/753,619, filed on Feb. 4, 2025, which is incorporated by reference herein in its entirety.

### FEDERALLY SPONSORED RESEARCH

This invention was made with government support under grant CMMI1747685 awarded by the National Science Foundation (NSF). The government has certain rights in the invention.

### BACKGROUND

Stretchable bioelectronics hold the key for creating implantable and on-skin devices that can conform to nonplanar and dynamic human body surfaces, which are indispensable for high-fidelity continuous monitoring of physiological signals and personalized healthcare. A major challenge in these emerging technologies is the significant mismatch between the properties of soft biological systems and stiff electronic devices. To realize stable integration with biological tissues with seamless conformal interfaces, it is crucial to ensure that the physiological deformation of soft tissues is not impeded by the bioelectronics and that the deformation of the tissues does not interfere or impair the functionality of the electronic devices.

To enhance the stretchability of electronic devices, considerable efforts have been devoted to the development of intrinsically stretchable materials that preserve electrical connectivity under extensive deformation. However, the electronic properties of these stretchable materials are generally inferior to the conventional non-stretchable materials, and their properties often drift during repeated stretching cycles. Alternatively, geometrical design strategies (e.g., serpentines and wrinkles) have been employed to enhance the stretchability of thin metal films by converting strain into structural bending, achieving high conductivity and stability within a specific strain limit. However, these designs demand specialized fabrication techniques for patterning on elastomeric substrates and extra space for the necessary bends and folds, potentially leading to larger devices and lower packing density of components, reducing areal efficiency. Additionally, the microscopic wrinkles often prevent the conformal interface that is critical for signal transduction.

Another strategy for stretchable electronics is to generate a nonuniform strain distribution by using substrates comprising strain-free and strain-absorbing regions. Recent developments in strain-engineered elastomeric substrates using island-bridge structures have been employed for stretchable electronics. Such designs include embedding stiff platforms within soft substrates, introducing soft interlayer, utilizing core/shell packages with ultralow modulus cores, and incorporating liquid-filled cavities in the soft substrate to protect functional components from strain. Nonetheless, these systems all involve stiff-soft interfaces with a sharp change of mechanical properties, which are prone to mechanical failures due to high stress concentrations. To this end, it is important to design interfaces that can bridge the gap between stiff components and soft surfaces, ensuring they can withstand strains without compromising performances.

What is needed is a universal gradient interface for super stretchable and strain-insensitive bioelectronic devices.

### SUMMARY

One embodiment described herein is a printed universal gradient interface for stretchable and strain-insensitive bioelectronics, the universal gradient interface comprising a multi-polymer gradient extending between a first polymer end and a second polymer end, the first polymer end comprising a first polymer content, the second polymer end comprising a second polymer content, and the multi-polymer gradient comprising a graded weight ratio of the first polymer content to the second polymer content of between 1:0 and 0:1 from the first polymer end to the second polymer end. In one aspect, each of the first polymer content and the second polymer content comprises a polyurethane polymer, a polyurethane dispersion (PUD) polymer, a thermoplastic polyurethane (TPU) polymer, a polynitrile polymer, a polyether polymer, a polycarbonate polymer, a polyalcohol polymer, a polyethylene polymer, a polyethylene oxide polymer, a polyethylene glycol polymer, a polypropylene polymer, a polyacrylate polymer, a polyester polymer, a polyvinyl polymer, a polyphosphazene polymer, a polysulfone polymer, a polysiloxane polymer, a polyamide polymer, a polylactic acid polymer, a polyacrylate polymer, a polyimide polymer, a fluoropolymer, or combinations thereof. In another aspect, the multi-polymer gradient comprises a polyurethane dispersion (PUD) gradient extending between a first PUD end and a second PUD end, the first PUD end comprising a first PUD content, the second PUD end comprising a second PUD content, and the multi-polymer gradient comprising a graded weight ratio of the first PUD content to the second PUD content of between 1:0 and 0:1 from the first PUD end to the second PUD end. In another aspect, each of the first PUD content and the second PUD content comprises one or more of a polyether-based PUD, a polyester-based PUD, or a polycarbonate-based PUD. In another aspect, the first PUD content comprises flexible polyester polyol segments and the second PUD content comprises stiff polyester polyol segments derived from methylene diphenyl diisocyanate (MDI). In another aspect, the first polymer content has a Young's modulus of about 0.05 MPa to about 80 MPa and the second polymer content has a Young's modulus of about 100 MPa to about 50 GPa. In another aspect, the multi-polymer gradient has a Young's modulus ranging from about 0.05 MPa at the first polymer end to about 50 GPa at the second polymer end. In another aspect, the universal gradient interface has a thickness of about 0.5 μm to about 2 mm. In another aspect, the second polymer end is configured to functionally connect to an electrically conductive material. In another aspect, the electrically conductive material comprises one or more of a metal, a semiconductor, a conductive polymer, or an electrode.

Another embodiment described herein is a printed bioelectronic device, comprising: a universal gradient interface comprising a multi-polymer gradient extending between a first polymer end and a second polymer end, the first polymer end comprising a first polymer content, the second polymer end comprising a second polymer content, and the multi-polymer gradient comprising a graded weight ratio of the first polymer content to the second polymer content of between 1:0 and 0:1 from the first polymer end to the second polymer end; and an electrically conductive material functionally connected to the second polymer end of the universal gradient interface. In one aspect, each of the first polymer content and the second polymer content comprises a polyurethane polymer, a polyurethane dispersion (PUD) polymer, a thermoplastic polyurethane (TPU) polymer, a polynitrile polymer, a polyether polymer, a polycarbonate polymer, a polyalcohol polymer, a polyethylene polymer, a polyethylene oxide polymer, a polyethylene glycol polymer, a polypropylene polymer, a polyacrylate polymer, a polyester polymer, a polyvinyl polymer, a polyphosphazene polymer, a polysulfone polymer, a polysiloxane polymer, a polyamide polymer, a polylactic acid polymer, a polyacrylate polymer, a polyimide polymer, a fluoropolymer, or combinations thereof. In another aspect, the multi-polymer gradient of the universal gradient interface comprises a polyurethane dispersion (PUD) gradient extending between a first PUD end and a second PUD end, the first PUD end comprising a first PUD content, the second PUD end comprising a second PUD content, and the multi-polymer gradient comprising a graded weight ratio of the first PUD content to the second PUD content of between 1:0 and 0:1 from the first PUD end to the second PUD end. In another aspect, the electrically conductive material comprises one or more of a metal, a semiconductor, a conductive polymer, or an electrode. In another aspect, the first polymer end of the universal gradient interface is configured to functionally connect to a biological surface. In another aspect, the biological surface comprises a mammalian tissue or organ. In another aspect, the mammalian tissue or organ comprises human skin or a human heart. In another aspect, the bioelectronic device is a wearable bioelectronic device. In another aspect, the bioelectronic device is a sensor comprising a temperature sensor, a photodetector, a pulse sensor, or an oximeter. In another aspect, the bioelectronic device exhibits improved strain insensitivity and resistivity changes under a high stretch ratio relative to conventional bioelectronic devices not having the universal gradient interface. In another aspect, the bioelectronic device withstands a uniaxial stretch ratio of up to about 180% without electrical failure. In another aspect, the bioelectronic device exhibits a relative resistance change of less than about 5% under a uniaxial stretch ratio of up to 100%.

Another embodiment described herein is a method of making a universal gradient interface for stretchable and strain-insensitive bioelectronics, the method comprising: mixing a first aerosolized ink stream comprising a first polymer and a second aerosolized ink stream comprising a second polymer using a carrier gas to form an aerosolized polymer mixture; and aerosol jet printing the aerosolized polymer mixture onto a substrate material using a sheath gas such that the first aerosolized ink stream comprising the first polymer is first deposited onto the substrate material to form a first polymer end comprising the first polymer; wherein flow rates of the first aerosolized ink stream and the second aerosolized ink stream are continuously adjusted to form a multi-polymer gradient extending between the first polymer end and a second polymer end comprising the second polymer, the multi-polymer gradient comprising a graded weight ratio of the first polymer to the second polymer of between 1:0 and 0:1 from the first polymer end to the second polymer end. In one aspect, the substrate material is treated with plasma to improve surface hydrophilicity prior to aerosol jet printing. In another aspect, the method further comprises drying the universal gradient interface to remove any solvent. In another aspect, the method further comprises aerosol jet printing a third aerosolized ink stream comprising an electrically conductive material onto the second polymer end, thereby functionally connecting the second polymer end to the electrically conductive material. In another aspect, the electrically conductive material comprises one or more of a metal, a semiconductor, a conductive polymer, or an electrode. In another aspect, aerosol jet printing comprises a sheath gas flow rate of about 50 sccm to about 70 sccm. In another aspect, aerosol jet printing comprises an aerosol jet print speed of about 1 mm/s to about 5 mm/s. In another aspect, each of the flow rates of the first aerosolized ink stream and the second aerosolized ink stream ranges from about 0 sccm to about 35 sccm. In another aspect, one or more of the first aerosolized ink stream and the second aerosolized ink stream further comprise one or more additives, solvents, or surfactants. In another aspect, the first polymer has a Young's modulus of about 0.05 MPa to about 80 MPa and the second polymer has a Young's modulus of about 100 MPa to about 50 GPa.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-C show stretchable devices enabled by universal gradient interfaces (UGIs) printed using aerosol-based multi-materials printing (AMMP). FIG. 1A shows a scheme for one-step AMMP to print both UGI and functional devices. FIG. 1B shows a schematic illustration of the original and stretched states of a 3D gradient UGI. Young's modulus distributions of the UGI and the maximum principal Lagrange strain map of the 3D UGI under 100% bi-axial stretch ratio. FIG. 1C shows a schematic illustration of the wide range of materials that can be integrated with the UGI.

FIG. 2A-C show simulations for each design for uniaxial stretch ratio of 100%. FIG. 2A shows a schematic of the simulation setup. FIG. 2B shows simulation dimensions with 3D in-plane gradient design as an example. FIG. 2C shows principal logarithmic strain distribution (top) and maximum principal stress distribution (bottom) of each design.

FIG. 3A-C show simulations for each design for bi-axial stretch ratio of 100%. FIG. 3A shows a schematic of the simulation setup. FIG. 3B shows simulation dimensions with 3D in-plane gradient design as an example. FIG. 3C shows principal logarithmic strain distribution (top) and maximum principal stress distribution (bottom) of each design.

FIG. 4A-B show X-Y-plane printing resolution. Printing of an individual line showing the X-Y plane resolution as small as 10 μm for soft polyurethane dispersion (PUD) (FIG. 4A) and hard PUD (FIG. 4B) inks (Nozzle size: 30 gauge).

FIG. 5A-B show Z-plane printing resolution. FIG. 5A shows a typical white light profilometer scan across the printed 1-layer PUD film showing the deposition resolution as small as 500 nm (nozzle size: 25 gauge). FIG. 5B shows the surface topology of the printed film.

FIG. 6 shows the design of each non-gradient and gradient pattern.

FIG. 7A-F show non-gradient and 1D out-of-plane UGI properties. FIG. 7A shows the printed pattern; FIG. 7B shows site-specific component ratio; and FIG. 7C shows the DIC under 100% stretch ratio for non-gradient design. FIG. 7D shows the printed pattern; FIG. 7E shows site-specific component ratio; and FIG. 7F shows the DIC under 100% stretch ratio for 1D out-of-plane gradient design.

FIG. 8 shows the DIC figures for each design at crack.

FIG. 9 shows FEA results under uniaxial tension.

FIG. 10 shows optical images of stretching process for each design.

FIG. 11 shows the tensile test for non-gradient and 3D UGI designs.

FIG. 12A-C show flash sintering of printed Au sensor. FIG. 12A shows ultra-fast flash sintering of the printed gold and the sintering condition. FIG. 12B-C show printed gold before (FIG. 12B) and after (FIG. 12C) flash sintering.

FIG. 13 shows the stability curve of the printed MXene-based sensor on soft PUD substrate is under 20% strain for 100 cycles.

FIG. 14A shows photographs of the UGI under unstretched and stretched states. FIG. 14B shows RRC vs. stretch ratio for gold-based sensor on a 3D gradient UGI.

FIG. 15A shows the design and fabrication process of the temperature sensor. FIG. 15B shows the curve of resistance of the sensor under different temperatures.

FIG. 16A shows photographs of the UGI under not humidified and humidified states. FIG. 16B shows RRC vs. stretch ratio for printed sensors under different humidified states.

FIG. 17 shows substrate transparency analysis.

FIG. 18 shows the optical sensor fabrication process.

FIG. 19 shows responsivity as a function of illumination power measured with a 405 nm laser under a bias voltage of 5 V.

FIG. 20A shows detailed response time and responsivity relationship. FIG. 20B shows a comparison of responsivity and response time in the devices in this work with previously reported all-printed photodetectors in the visible light range.

FIG. 21A shows a schematic illustration of the cross-sectional view of the pressure-tunable cylindrical cavity. FIG. 21B shows an optical image of the pressure-tunable cylindrical cavity connected with a mini pump.

FIG. 22A shows a schematic illustration of dimension for simulation. FIG. 22B shows modulus distribution of the sample. FIG. 22C shows strain distribution during stretching.

FIG. 23A shows a 3D photodetector array location coding. FIG. 23B-C show photocurrent of printed 3D photodetector array under normally-incident illumination (FIG. 23B) and under obliquely incident light (FIG. 23C).

FIG. 24 shows the connection setup for the skin-mounted sensors to the external measurement equipment.

FIG. 25A shows the composition of AC and DC components in the PPG waveform. FIG. 25B shows schematic representations of both original and processed signals for red (650 nm) and green (530 nm) light. FIG. 25C shows a processing diagram used to mitigate the influence of DC components. FIG. 25D shows a graph of matching R_{OS} values with SO₂.

FIG. 26 shows an oximeter printed on UGI and soft substrate before and after on-skin uniaxial stretching. Scale bar, 2.5 mm.

FIG. 27A-M show investigation of different UGI designs. Gradient design, maximum principal Lagrange strain map (Ep) under 100% uniaxial stretch ratio, and optical image under maximum stretch ratio in 1D in-plane gradient (FIG. 27A-C), 2D in-plane gradient (FIG. 27D-F), and 3D gradient (FIG. 27G-I). FIG. 27J shows composition and modulus distributions vs. X-positions for 1D and 2D in-plane gradient. FIG. 27K shows composition and modulus distributions vs. Z-positions for 3D gradient. FIG. 27L shows maximum principal Lagrange strain values on the center of the stiff region vs. external stretch for different UGI designs. FIG. 27M shows tensile test results for different UGI designs.

FIG. 28A-J illustrate the strain-insensitive effect of various conductive materials printed on the UGI. Relative resistance change (RRC) vs. stretch ratio for PEDOT:PSS (FIG. 28A); Au (FIG. 28B); AgNWs (FIG. 28C); and MXene (FIG. 28D). Scale bar, 100 μm. FIG. 28E shows comparison of RRC in this work with other stretchable electronics. FIG. 28F shows temperature sensor printed on the UGI. FIG. 28G shows a strain sensor printed on soft polyurethane. Scale bar, 4 mm. FIG. 28H shows as schematic and FIG. 28I shows an optical image of multimodal temperature and strain sensor on an artificial heart model. FIG. 28J shows real-time strain and temperature sensing during the periodic diastole and systole when the heart model is under stable temperature followed by a transient heating.

FIG. 29A-M show stretchable photodetectors. FIG. 29A shows a schematic and FIG. 29B shows optical images and AFM showing a printed photodetector on the UGI. FIG. 29C shows photocurrent vs. bias voltage under different illumination powers. FIG. 29D shows time-dependent photocurrent at an applied DC bias of 5 V. Dark current vs. bias voltage under different stretch ratios on soft polyurethane (FIG. 29E), and UGI (FIG. 29F). FIG. 29G shows photocurrent vs. bias voltage under different illumination powers when the UGI is under no stretch and 25% stretch. FIG. 29H shows a schematic of a deformable 3D photodetector array. FIG. 29I-J show optical images of the 3D photodetector under flat and inflated state. FIG. 29K shows dark current vs. bias voltage during flat and inflated state. FIG. 29L-M show the photocurrent mapping under upright illumination and tilted-angle illumination. The radius of the hemisphere is 30 mm. Scale bar, 5 mm.

FIG. 30A-H show printed wearable multimodal sensors for health monitoring. FIG. 30A shows an optical image of the multimodal sensor. FIG. 30B shows optical images of the temperature sensor, pulse sensor, and oximeter consisting of a printed photodetector (PD) and a green LED and a red LED. Scale bar, 2 mm. FIG. 30C-D show characteristics of the temperature sensor (FIG. 30C) and pulse sensor (FIG. 30D). FIG. 30E shows motion artifacts for oximeter printed on UGI and soft substrate. FIG. 30F shows a schematic of the oximeter in reflectance mode. FIG. 30G shows molar extinction coefficient of oxygenated (orange solid line) and deoxygenated (blue dashed line) hemoglobin in arterial blood, and absorptivity of oximeter vs. wavelength. FIG. 30H shows an output signal from oximeter with 99% and 96% oxygenation of blood. The green and red lines represent the signals when the green and red LEDs are operated.

### DETAILED DESCRIPTION

The present disclosure relates generally to the field of stretchable bioelectronics, and more particularly to universal gradient interfaces (UGIs) that bridge the mechanical property gap between soft biological tissues and stiff electronic materials. Soft biological tissues typically have a modulus ranging from a few kPa to a few MPa, whereas most electronic materials have a modulus ranging from hundreds of MPa to GPa. This significant mismatch in mechanical properties presents challenges for stable integration of electronic devices with biological systems.

In various aspects, the disclosed UGIs comprise a multi-polymer gradient having a gradient modulus distribution that transitions from a soft region to a stiff region. The gradient modulus distribution may span at least two orders of magnitude, enabling effective strain isolation at the stiff region where electronic components may be disposed to generate bioelectronic devices. The disclosed UGIs and bioelectronic devices may be printed using aerosol-based multi-materials printing (AMMP), which allows precise spatial control over local material compositions and properties with submicron resolution. The gradient may extend in one, two, or three dimensions, with 3D gradient designs providing optimal strain distribution and mechanical performance. The UGI may comprise various polymers with graded weight ratios transitioning from soft/flexible polymer content to hard/stiff polymer content based on the elastic (Young's) moduli of the different polymers.

In various aspects, a printed UGI for stretchable and strain-insensitive bioelectronics is disclosed. The UGI may comprise a multi-polymer gradient extending between a first polymer end and a second polymer end with a graded weight ratio from 1:0 to 0:1. The disclosed UGI may connect a biological surface to an electrically conductive material, enabling improved strain insensitivity and electrical stability under high stretch ratios. Methods of making the UGI via aerosol jet printing techniques and bioelectronic devices incorporating the UGI are also disclosed.

In various aspects, the disclosed UGI enables wearable or implantable bioelectronic devices for health sensing and monitoring applications. In some aspects, multimodal devices and sensors including temperature sensors, pulse sensors, photodetectors, and oximeters may be integrated on the UGI for on-skin health monitoring of body temperature, heart rate, and blood oxygen saturation levels. The disclosed printed UGIs and bioelectronic devices may conformally and functionally attach to biological surfaces such as human skin or dynamic tissue and organ surfaces while maintaining strain-insensitive electronic functionality during physiological deformation.

Unless otherwise defined, all technical and scientific terms used herein have the same meaning as commonly understood by one of ordinary skill in the art. For example, any nomenclatures used in connection with, and techniques of chemistry, microelectronics, and biochemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed inventions. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points, or as described above in the definition of “about.”

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30 °C, including all integers and endpoints within the specified range. In another aspect, “room temperature” refers a temperature of about 15-30 °C; about 20-30 °C; about 22-30 °C; about 25-30 °C; about 27-30 °C; about 15-22 °C; about 15-25 °C; about 15-27 °C; about 20-22 °C; about 20-25 °C; about 20-27 °C; about 22-25 °C; about 22-27 °C; about 25-27 °C; about 15 °C.±10%; about 20 °C±10%; about 22 °C±10%; about 25 °C.±10%; about 27 °C.±10%; ~20 °C, ~22 °C, ~25 °C, or ~27 °C, at standard atmospheric pressure.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “universal gradient interface” or “UGI” refers to a printed gradient interface comprising a multi-polymer gradient with a gradient modulus distribution that transitions from a soft polymer region to a stiff polymer region. The multi-polymer gradient comprises graded weight ratios of different polymer contents, transitioning from a first polymer end to a second polymer end. The UGI acts as a functional interface that bridges the mechanical property gap between soft biological tissues and stiff electronic materials by providing a gradual transition in modulus properties across the multi-polymer gradient.

### Printed Universal Gradient Interfaces

Described herein are printed universal gradient interfaces (UGIs) for stretchable and strain-insensitive bioelectronics comprising a multi-polymer gradient extending between a first polymer end and a second polymer end. The first polymer end comprises a first polymer content, and the second polymer end comprises a second polymer content. The multi-polymer gradient comprises a graded weight ratio of the first polymer content to the second polymer content of between 1:0 and 0:1 from the first polymer end to the second polymer end.

Various combinations of different polymers having different elastic (Young's) modulus properties may be used in the disclosed UGIs. In various aspects, each of the first polymer content and the second polymer content may comprise a polyurethane polymer, a polyurethane dispersion (PUD) polymer, a thermoplastic polyurethane (TPU) polymer, a polynitrile polymer, a polyether polymer, a polycarbonate polymer, a polyalcohol polymer, a polyethylene polymer, a polyethylene oxide polymer, a polyethylene glycol polymer, a polypropylene polymer, a polyacrylate polymer, a polyester polymer, a polyvinyl polymer, a polyphosphazene polymer, a polysulfone polymer, a polysiloxane polymer, a polyamide polymer, a polylactic acid polymer, a polyacrylate polymer, a polyimide polymer, a fluoropolymer, or combinations thereof. Non-limiting exemplary polymers include polyolefins (e.g., polyethylenes, poly(butene-1), poly(n-pentene-2), polypropylene, polytetrafluoroethylene), polyamines (e.g., poly(ethylene imine) and polypropylene imine (PPI)); polyamides (e.g., polyamide (Nylon), poly(E-caprolactam) (Nylon 6), poly(hexamethylene adipamide) (Nylon 66)), polyimides (e.g., polyimide, polynitrile, poly(acrylonitrile) (PAN), and poly(pyromellitimide-1,4-diphenyl ether) (Kapton®) (NOMEX®) (KEVLAR®)); polyether ether ketone (PEEK); vinyl polymers (e.g., polyacrylamide, poly(2-vinyl pyridine), poly(N-vinylpyrrolidone), poly(methylcyanoacrylate), poly(ethylcyanoacrylate), poly(butylcyanoacrylate), poly(isobutylcyanoacrylate), poly(vinyl acetate), poly(vinyl alcohol), poly(vinyl chloride), poly(vinyl fluoride), poly(-vinyl pyridine), vinyl polymer, polychlorotrifluoro ethylene, and poly(isohexylcynaoacrylate)); polyacetals; polyesters (e.g., polycarbonate, polybutylene terephthalate, polyhydroxybutyrate); polyethers (poly(ethylene oxide) (PEO), poly(propylene oxide) (PPO), poly(tetramethylene oxide) (PTMO)); vinylidene polymers (e.g., polyisobutylene, poly(methyl styrene), poly(methylmethacrylate) (PMMA), poly(vinylidene chloride), and poly(vinylidene fluoride)); polyaramides (e.g., poly(imino-1,3-phenylene iminoisophthaloyl) and poly(imino-1,4-phenylene iminoterephthaloyl)); polyheteroaromatic compounds (e.g., polybenzimidazole (PBI), polybenzobisoxazole (PBO) and polybenzobisthiazole (PBT)); polyheterocyclic compounds (e.g., polypyrrole); polyurethanes and polyurethane dispersions (PUDs); phenolic polymers (e.g., phenol-formaldehyde); polyalkynes (e.g., polyacetylene); polydienes (e.g., 1,2-polybutadiene, cis or trans-1,4-polybutadiene); polysiloxanes (e.g., poly(dimethylsiloxane) (PDMS), poly(diethylsiloxane) (PDES), polydiphenylsiloxane (PDPS), and polymethylphenylsiloxane (PMPS)); and inorganic polymers (e.g., polyphosphazene, polyphosphonate, polysilanes, polysilazanes). Additional non-limiting examples of suitable polymers having different ranges of moduli include PDMS (Sylgard® 184): ~0.5-3 MPa; Ecoflex®: ~0.05-0.2 MPa; Dragon Skin® (silicone rubber elastomer): ~0.3-1 MPa; thermoplastic polyurethane (TPU) (soft grades): ~5-50 MPa; polyethylene and low-density polyethylene (LDPE): ~200-400 MPa; polypropylene (PP): ~1-1.5 GPa; polyurethane (rigid grades): ~100-800 MPa; nylons (PA6, PA12): ~1-3 GPa; polylactic acid (PLA): ~2-3.5 GPa; polyethylene terephthalate (PET): ~2-3 GPa; PMMA (acrylic): ~2-3 GPa; polycarbonate (PC): ~2-2.5 GPa; acrylonitrile butadiene styrene (ABS): ~1.5-2.5 GPa; polyetheretherketone (PEEK): ~3-4 GPa; polyimide (Kapton®): ~2.5-4 GPa; liquid crystal polymers (LCPs): ~5-20 GPa; epoxy resins (filled): ~5-50 GPa; and carbon-fiber-reinforced polymers: ~50-150 GPa.

In certain non-limiting exemplary embodiments disclosed herein, the first polymer content comprises a first polyurethane dispersion (PUD) content and the second polymer content comprises a second PUD content, where the first PUD content and the second PUD content have different Young's moduli. In certain embodiments, each of the first polymer content and the second polymer content comprises one or more of a polyether-based PUD, a polyester-based PUD, or a polycarbonate-based PUD. In certain embodiments, a first PUD content comprises flexible polyester polyol segments with low cross-linking, resulting in a lower modulus and higher elongation at break. In certain embodiments, a second PUD content comprises stiff polyester polyol segments derived from methylene diphenyl diisocyanate (MDI), leading to a higher modulus and lower elongation at break. Alternative isocyanates may include, for example, toluene diisocyanate (TDI), hexamethylene diisocyanate (HDI), isophorone diisocyanate (IPDI), or combinations thereof.

In various aspects, the weight ratio of the first polymer content to the second polymer content at the first polymer end may be about 100:0, about 99:1, about 95:5, about 90:10, or about 85:15. In various aspects, the weight ratio of the first polymer content to the second polymer content at the second polymer end may be about 0:100, about 1:99, about 5:95, about 10:90, or about 15:85. In some aspects, the weight ratio of the first polymer content to the second polymer content at an intermediate position between the first polymer end and the second polymer end may be, for example, about 75:25, about 70:30, about 60:40, about 50:50, about 40:60, about 30:70, or about 25:75. In some embodiments, the graded weight ratio of first polymer content to second polymer content transitions continuously from about 100:0 at the first polymer end to about 0:100 at the second polymer end. In some embodiments, the graded weight ratio transitions from about 90:10 at the first polymer end to about 10:90 at the second polymer end. In some embodiments, the graded weight ratio transitions from about 80:20 at the first polymer end to about 20:80 at the second polymer end.

In various aspects, the first polymer content has a Young's modulus that is less than the Young's modulus of the second polymer content. In various aspects, the first polymer content has a Young's modulus of about 0.05 MPa to about 80 MPa. For example, the first polymer content may have a Young's modulus of about 0.1 MPa to about 70 MPa, about 0.5 MPa to about 60 MPa, about 1 MPa to about 50 MPa, or about 5 MPa to about 40 MPa. In some aspects, the first polymer content may have a Young's modulus of less than about 0.05 MPa or greater than about 80 MPa. In some embodiments, the first polymer content may have a Young's modulus of about 0.5 MPa to about 10 MPa, or about 1 MPa to about 5 MPa. In other embodiments, the first polymer content has a Young's modulus of about 0.05 MPa to about 1 MPa. In various aspects, the second polymer content has a Young's modulus of about 100 MPa to about 50 GPa. For example, the second polymer content may have a Young's modulus of about 200 MPa to about 40 GPa, about 500 MPa to about 30 GPa, about 1 GPa to about 20 GPa, or about 5 GPa to about 10 GPa. In some aspects, the second polymer content may have a Young's modulus of less than about 100 MPa or greater than about 50 GPa. For instance, the second polymer content may have a Young's modulus ranging from about 80 MPa to about 150 MPa or from about 50 GPa to about 150 GPa. In some embodiments, the second polymer content may have a Young's modulus of about 100 MPa to about 5 GPa, about 100 MPa to about 10 GPa, or about 500 MPa to about 2 GPa. In other embodiments, the second polymer content has a Young's modulus of about 1 GPa to about 50 GPa. In certain non-limiting exemplary embodiments disclosed herein, a first PUD polymer content may have a Young's modulus of about 0.5 MPa to about 5 MPa, and a second PUD polymer content may have a Young's modulus of about 50 MPa to about 150 MPa.

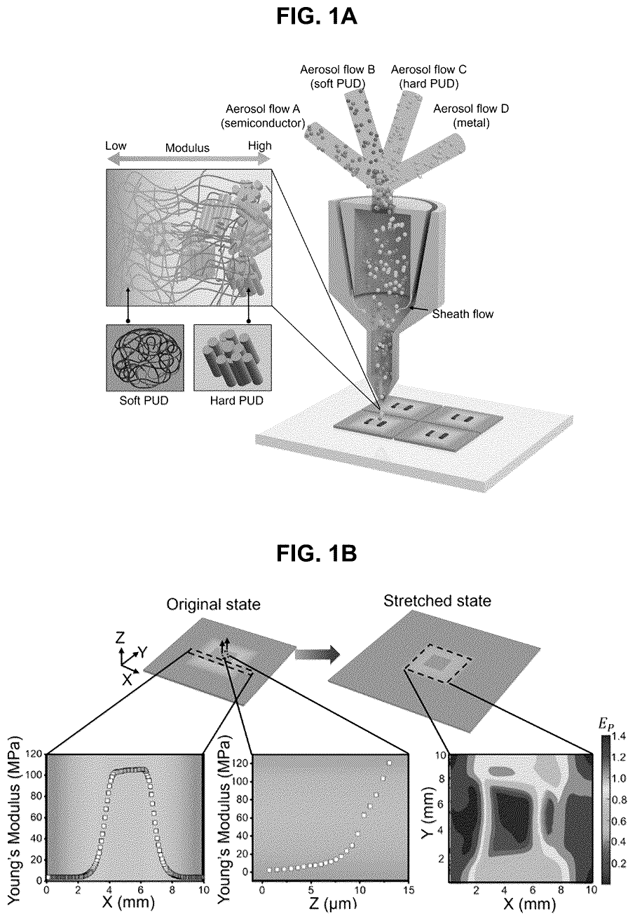

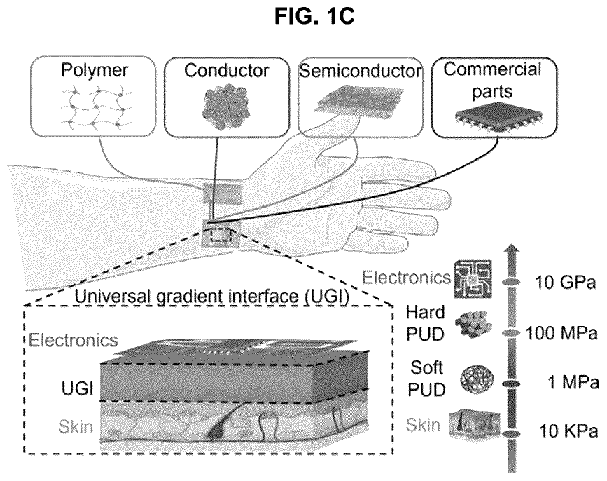

In various aspects, the UGI comprises a gradient modulus distribution spanning at least two orders of magnitude from one polymer end to the other. For example, in some aspects, the multi-polymer gradient of the UGI has a Young's modulus ranging from about 0.05 MPa at the first polymer end to about 50 GPa at the second polymer end. In certain non-limiting exemplary embodiments disclosed herein, the multi-polymer gradient of the UGI may have a Young's modulus ranging from about 1-10 MPa at a soft polymer end to about 100 MPa-10 GPa at a hard polymer end (e.g., see FIG. 1B-C). The gradient modulus distribution of the UGI gradually transitions from a soft/flexible region at the first polymer end to a stiff/rigid region at the second polymer end, reducing strain at interfaces between the soft region and the stiff region during mechanical deformation. In some aspects, the gradient modulus distribution may extend in one or more dimensions. For example, the gradient may extend in an X-direction, a Y-direction, a Z-direction, or combinations thereof. The disclosed UGI configurations may comprise a 3D gradient design. This 3D gradient design may systematically alter the modulus across all three dimensions: X, Y, and Z. In some embodiments, the gradient modulus distribution extends in three dimensions comprising the X-direction, a Y-direction, and a Z-direction.

In various aspects, the UGI may have different thicknesses depending on the specific application. In some aspects, the UGI may have a thickness of about 0.05 μm to about 2 mm. In some embodiments, the UGI may have a thickness of about 0.05 μm to about 100 μm, about 0.1 μm to about 50 μm, about 0.2 μm to about 25 μm, or about 0.3 μm to about 20 μm. In some embodiments, the UGI may have a thickness of about 0.1 μm to about 2 μm, about 0.1 μm to about 5 μm, about 0.1 μm to about 10 μm, about 0.5 μm to about 10 μm, about 0.5 μm to about 15 μm, or about 0.5 μm to about 20 μm. In other embodiments, the UGI may have a thickness of about 1 μm to about 10 μm, about 1 μm to about 8 μm, or about 2 μm to about 6 μm. In further embodiments, the UGI may have a thickness of about 50 μm to about 500 μm, about 100 μm to about 1 mm, or about 0.5 mm to about 2 mm. In certain aspects, the UGI may have a thickness of about 1 μm, about 5 μm, about 10 μm, about 15 μm, about 20 μm, about 25 μm, about 100 μm, about 500 μm, about 1 mm, or about 2 mm.

In various aspects, the second polymer end (i.e., stiff region) of the UGI is configured to functionally connect to an electronic device, optoelectronic device, sensor, battery, or any electrically conductive material. Suitable electrically conductive materials may include, for example, one or more of a metal (e.g., gold, copper, silver, molybdenum, MXene), semiconductor, conductive polymer, or electrode.

In some aspects, the UGI exhibits superior strain isolation performance. For example, the strain-isolation region may exhibit a maximum principal strain of less than about 5% when the UGI is subjected to a stretch ratio of about 50% or greater. In some embodiments, the strain-isolation region may exhibit a maximum principal strain of less than about 3% when the UGI is subjected to a 100% uniaxial stretch. In some embodiments, the strain-isolation region exhibits a maximum principal strain of less than about 2.5% when the UGI is subjected to a 100% equi-biaxial stretch (FIG. 1B). Under 100% uniaxial tension, maximum principal strains may remain as low as about 1.1% for 3D gradients in stiff areas. In certain exemplary embodiments, the 3D UGI may exhibit strain in the stiff region as small as about 2.7% while the uniaxial stretch ratio approaches about 180%.

In some aspects, the UGI exhibits cyclic durability. In some embodiments, the UGI may maintain its strain-isolation performance over about 100 cycles, about 500 cycles, or about 1000 cycles of repeated stretching. In some embodiments, the UGI maintains stable electrical properties in integrated devices over 1000 cycles at a 50% stretch ratio, with a relative resistance change (RRC) of less than about 1.6% throughout the cycles. In some embodiments, the UGI maintains consistent performance with minimal RRC change of less than about 1.3% during stretching at 50% stretch ratio under different humidity conditions.

In various aspects, the first polymer end (i.e., soft region) of the UGI may be configured to functionally connect to a biological surface. Suitable biological surfaces may include mammalian tissues or organs. In some embodiments, the biological surface comprises human skin. In some embodiments, the biological surface comprises a human heart or other dynamic tissue or organ surface. The UGI may conformally attach to the biological surface while maintaining strain-insensitive electronic functionality during physiological deformation.

In various aspects, the UGI may be printed on various substrate materials. Suitable substrate materials may include elastomeric substrates, flexible polymer substrates, or biocompatible substrates. The substrate may be treated with plasma to improve surface hydrophilicity prior to printing.

### Printed Bioelectronic Devices

Also described herein are printed bioelectronic devices comprising a UGI comprising a multi-polymer gradient extending between a first polymer end and a second polymer end, the first polymer end comprising a first polymer content, the second polymer end comprising a second polymer content, and the multi-polymer gradient comprising a graded weight ratio of the first polymer content to the second polymer content of between 1:0 and 0:1 from the first polymer end to the second polymer end; and an electrically conductive material functionally connected to the second polymer end of the UGI.

In various aspects, the electrically conductive material may include, for example, one or more of a metal (e.g., gold, copper, silver, molybdenum, MXene), semiconductor, conductive polymer, or electrode. In some aspects, the electrically conductive material comprises a conductive polymer. Suitable conductive polymers may include, for example, poly(3,4-ethylenedioxythiophene) polystyrene sulfonate (PEDOT:PSS), polyaniline (PANI), polypyrrole (PPy), polythiophene, poly(3-hexylthiophene) (P3HT), or combinations thereof. In certain non-limiting exemplary embodiments, the conductive polymer comprises PEDOT: PSS. The electrically conductive material may take the form of microscale or nanoscale rods, tubes, fibers, wires, particles, or flakes (e.g., microtubes or nanotubes, microfibers or nanofibers, nanowires, nanoparticles, etc.). In some embodiments, the electrically conductive material may have a particle size in the range of about 1 nm to about 100 μm, or from about 100 nm to about 10 μm.

In various embodiments, the electrically conductive material exhibits a Young's modulus greater than the second polymer end (stiff polymer end region) of the universal gradient interface. For example, the electrically conductive material may exhibit a Young's modulus of greater than about 100 MPa, greater than about 250 MPa, greater than about 500 MPa, greater than about 1 GPa, greater than about 10 GPa, greater than about 25 GPa, greater than about 50 GPa, or greater than about 100 GPa. The electrically conductive material may also exhibit a Young's modulus of at least about 1 GPa, at least about 10 GPa, at least about 50 GPa, or at least about 100 GPa. For example, the electrically conductive material may exhibit a Young's modulus from about 1 GPa to about 5 GPa, from about 5 GPa to about 10 GPa, from about 10 GPa to about 25 GPa, from about 25 GPa to about 50 GPa, from about 50 GPa to about 75 GPa, from about 75 GPa to about 100 GPa, from about 100 GPa to about 150 GPa, or from about 150 GPa to about 500 GPa. In some embodiments, polymer-based electrically conductive materials may exhibit moduli in the range of about 1-5 GPa, while metallic electrically conductive materials such as copper, gold, or aluminum may exhibit higher moduli in the range of about 50-200 GPa.

A variety of semiconducting and metallic 2D/1D/0D nanomaterials can be printed on the UGI to form bioelectronics with remarkable strain insensitivity and negligible resistivity changes under a high stretch ratio of up to 180%. The all-printed devices deliver high signal quality and near-perfect immunity to motion artifacts for on-skin health monitoring of blood oxygen saturation levels, temperature, and pulses. This facile approach greatly simplifies the fabrication of super-stretchable bioelectronics, enabling harmonious integration of a broad range of functional devices with biological systems for emerging applications such as wearable/implantable devices, soft/hybrid robotics, and human/machine interfaces, etc.

In some aspects, the electrically conductive material comprises zero-dimensional (0D) nanomaterials. Suitable 0D nanomaterials may include, for example, metal nanoparticles such as gold nanoparticles, silver nanoparticles, copper nanoparticles, platinum nanoparticles, palladium nanoparticles, or combinations thereof. The metal nanoparticles may have a diameter in a broad range of about 1 nm to about 100 nm. In a narrower aspect, the metal nanoparticles may have a diameter of about 5 nm to about 50 nm. In some embodiments, the metal nanoparticles have a diameter of about 10 nm to about 30 nm.

In some aspects, the electrically conductive material comprises one-dimensional (1D) nanomaterials. Suitable 1D nanomaterials may include, for example, nanowires such as silver nanowires (AgNWs), gold nanowires, copper nanowires, or combinations thereof. The nanowires may have a length in a broad range of about 1 μm to about 100 μm. In one aspect, the nanowires may have a length of about 5 μm to about 50 μm. The nanowires may have a diameter in a range of about 10 nm to about 200 nm or about 20 nm to about 100 nm. Suitable 1D nanomaterials may also include carbon nanotubes, including single-walled carbon nanotubes (SWCNTs), multi-walled carbon nanotubes (MWCNTs), or combinations thereof.

In some aspects, the electrically conductive material comprises two-dimensional (2D) nanomaterials. Suitable 2D nanomaterials may include, for example, MXene, graphene, reduced graphene oxide, transition metal dichalcogenides (TMDCs), or combinations thereof. Suitable MXene materials may include Ti₃C₂Tₓ, Ti₂CTₓ, V₂CTₓ, Nb₂CTₓ, or combinations thereof. Suitable TMDCs may include MoS₂, WS₂, MoSe₂, WSe₂, or combinations thereof. In certain non-limiting exemplary embodiments, the 2D nanomaterial comprises MoS₂ for photodetector applications.

In some aspects, the electrically conductive material comprises a metal. Suitable metals may include, for example, gold, silver, copper, platinum, aluminum, nickel, titanium, MXene, or combinations thereof. In some embodiments, the metal may comprise gold for temperature sensor applications. In some aspects, the electrically conductive material comprises a semiconductor. Suitable semiconductors may include, for example, transition metal dichalcogenides, metal oxides such as zinc oxide (ZnO), indium tin oxide (ITO), or indium gallium zinc oxide (IGZO), organic semiconductors, or combinations thereof.

In some aspects, the electrically conductive material comprises a material for a sensor. Suitable sensors may include, for example, temperature sensors, photodetectors, pulse sensors, oximeters, strain sensors, pressure sensors, humidity sensors, chemical sensors, biosensors, or combinations thereof. In some embodiments, the bioelectronic devices disclosed herein may comprise a single sensor or a plurality of sensors for multimodal sensing applications.

In various aspects, the bioelectronic device exhibits improved strain insensitivity and resistivity changes under a high stretch ratio relative to conventional bioelectronic devices not having the universal gradient interfaces disclosed herein. In one aspect, the device may exhibit a relative resistance change (ΔR/R0) of less than about 10% under a uniaxial stretch ratio of up to about 100%. In another aspect, the device may exhibit a relative resistance change of less than about 5% under a uniaxial stretch ratio of up to about 100%. In certain embodiments, the device may exhibit a relative resistance change of less than about 4.2% under a uniaxial stretch ratio of up to about 100%. In certain embodiments, the device may exhibit a relative resistance change of less than about 1.6% under cyclic loading at a 50% stretch ratio over 1000 cycles.

In various aspects, the device is capable of withstanding high stretch ratios without electrical failure. In one aspect, the device may withstand a uniaxial stretch ratio of up to about 100% without electrical failure. In another aspect, the device may withstand a uniaxial stretch ratio of up to about 150% without electrical failure. In another aspect, the device withstands a uniaxial stretch ratio of up to about 180% without electrical failure.

In various aspects, the first polymer end of the universal gradient interface of the device is configured to functionally connect to a biological surface. Suitable biological surfaces may include mammalian tissues or organs. In some embodiments, the biological surface comprises human skin. In some embodiments, the biological surface comprises a human heart or other dynamic organ surface. The bioelectronic device may conformally attach to the biological surface while maintaining strain-insensitive electronic functionality during physiological deformation.

In various aspects, the biological surface exhibits a Young's modulus less than the first polymer end (soft polymer end region) of the universal gradient interface. The biological surface may exhibit a Young's modulus of less than about 1 MPa or less than about 500 kPa. For example, the biological surface may exhibit a Young's modulus from about 1 kPa to about 10 kPa, from about 10 kPa to about 25 kPa, from about 25 kPa to about 50 kPa, from about 50 kPa to about 100 kPa, from about 100 kPa to about 200 kPa, from about 200 kPa to about 300 kPa, from about 300 kPa to about 400 kPa, and from about 400 kPa to about 500 kPa.

In various aspects, the bioelectronic device is a wearable bioelectronic device. In other aspects, the bioelectronic device is an implantable bioelectronic device. In some embodiments, the bioelectronic device may be configured for on-skin health monitoring applications including, for example, monitoring of body temperature, heart rate, and/or blood oxygen saturation levels. In some embodiments, the bioelectronic device comprises a wearable health monitoring system with a plurality of sensors for multimodal health monitoring.

In various aspects, the bioelectronic devices may be fabricated using aerosol-based multi-materials printing (AMMP). The AMMP technique enables one-step fabrication of both the UGI and electrically conductive material components in a single nozzle, significantly improving fabrication efficiency compared to conventional multi-step fabrication methods. Electrically conductive materials may be printed directly onto the stiff polymer end of the UGI using the same AMMP system used to print the UGI. A variety of electrically conductive materials may be printed on the UGI, including, for example, metals such as gold nanoparticles, semiconductors such as MoS₂, conductive polymers such as PEDOT: PSS, and nanomaterials such as silver nanowires (AgNWs) and MXene. This all-printed fabrication approach greatly simplifies the manufacturing of stretchable bioelectronics, enabling precise control over device structures and local properties with submicron spatial resolution while maintaining compatibility with a broad range of functional materials.

### Methods of Making Universal Gradient Interfaces and Bioelectronic Devices

Also described herein are methods of making a universal gradient interface (UGI) for stretchable and strain-insensitive bioelectronics. The methods utilize aerosol-based multi-materials printing (AMMP) techniques that enable precise spatial control over local material compositions and properties with submicron resolution, allowing for UGIs and functional electronic materials to be aerosol jet printed in a single nozzle with high fabrication efficiency.

As shown in the example schematics of FIG. 1A-B, the UGI with a submicron stiffness gradient can be printed using AMMP techniques by in situ modulating the mixing ratio of multiple polymer inks of vastly different moduli properties in a single nozzle, realizing precise control of the local properties along all the three dimensions. This unique capability for in situ aerosol-phase mixing and printing enables instantaneous tuning of material compositions, a feature difficult to achieve with conventional multi-material printing methods that rely on liquid-liquid or solid-solid feedstocks.

In various aspects, the methods comprise mixing a first aerosolized ink stream comprising a first polymer (soft polymer ink) and a second aerosolized ink stream comprising a second polymer (hard polymer ink) using a carrier gas to form an aerosolized polymer mixture; and aerosol jet printing the aerosolized polymer mixture onto a substrate material using a sheath gas such that the first aerosolized ink stream comprising the first polymer is first deposited onto the substrate material to form a first polymer end comprising the first polymer. Flow rates of the first aerosolized ink stream and the second aerosolized ink stream are continuously adjusted to form a multi-polymer gradient extending between the first polymer end and a second polymer end comprising the second polymer, the multi-polymer gradient comprising a graded weight ratio of the first polymer to the second polymer of between 1:0 and 0:1 from the first polymer end to the second polymer end.

In some aspects, the method may comprise atomizing two or more inks into aerosols that include microscale droplets. The inks may be atomized via ultrasonication before being transferred to a printhead. In some aspects, an ultrasonic atomizing voltage may be about 30 V to about 50 V for both soft polymer inks and hard polymer inks having relatively lower and higher moduli, respectively. In some aspects, the inks may be primed under sonication for about 15 minutes to about 30 minutes prior to atomization to avoid aggregation and ensure uniform dispersion. The aerosols may then be mixed in situ in a single nozzle and focused using a co-flowing sheath gas prior to deposition. The AMMP technique enables precise control over local material composition by dynamically modulating the mixing ratio of different aerosolized inks during deposition.

In some aspects, the substrate material is treated with plasma to improve surface hydrophilicity prior to aerosol jet printing. Suitable substrate materials may include elastomeric substrates, flexible polymer substrates, or biocompatible substrates. During the printing process, a heating stage may be used to heat the substrate material and evaporate the ink solvents to minimize undesired drying effects.

In some aspects, the printing method further comprises aerosol jet printing a third aerosolized ink stream comprising an electrically conductive material onto the second polymer end, thereby functionally connecting the second polymer end to the electrically conductive material. In some embodiments, the electrically conductive material may comprise one or more of a metal, a semiconductor, a conductive polymer, or an electrode.

In some aspects, the printing method further comprises flash sintering the deposited electrically conductive material using, for example, a xenon flash lamp. Flash sintering may be used to achieve desired microstructures and properties for metal nanoparticle-based electronic components. In one aspect, the flash lamp voltage may be about 1.5 kV to about 3.0 kV. In another aspect, the flash lamp voltage may be about 2.0 kV to about 2.4 kV. In certain non-limiting exemplary embodiments, the flash lamp voltage is about 2.0 kV for gold nanoparticles or about 2.4 KV for MoS₂. In some aspects, the flash sintering pulse duration may be about 500 μs to about 1500 μs. In some embodiments, the flash sintering pulse duration is about 800 μs. In some aspects, the number of consecutive pulses may be about 1 to about 5. In some aspects, the pulse delay between consecutive pulses may be about 500 ms to about 2000 ms. The total sintering time may be less than about 2 seconds.

In various aspects, the aerosol jet printing process parameters may be adjusted to achieve desired printing resolution and gradient characteristics. In one aspect, the sheath gas flow rate may range from about 40 sccm to about 100 sccm, such as, for example, about 50 sccm to about 70 sccm. In certain non-limiting exemplary embodiments, the sheath gas flow rate is about 60 sccm. In some aspects, each of the flow rates of the first aerosolized ink stream and the second aerosolized ink stream may range from about 0 sccm to about 50 sccm. In some aspects, each of the flow rates may range from about 0 sccm to about 35 sccm. In some embodiments, the soft polymer ink flow rate ranges from about 10 sccm to about 30 sccm, and the hard polymer ink flow rate ranges from about 17 sccm to about 30 sccm. In some aspects, the aerosol jet print speed may be about 0.5 mm/s to about 10 mm/s. In some aspects, the aerosol jet print speed may be about 1 mm/s to about 5 mm/s. In certain non-limiting exemplary embodiments, the aerosol jet print speed is about 2 mm/s.

In some aspects, one or more of the first aerosolized ink stream and the second aerosolized ink stream may further comprise one or more additives, solvents, co-solvents, or surfactants. Suitable additives, solvents, co-solvents, or surfactants may include, for example, water, ethylene glycol (EG), dimethyl sulfoxide (DMSO), or combinations thereof. In some embodiments, a small amount of isopropyl alcohol (IPA) may be added as a defoamer to suppress foam formation during ultrasonic atomizing. In some aspects, the printing method further comprises drying the universal gradient interface to remove any solvent. The drying may be performed at room temperature or at elevated temperatures. In some embodiments, the heating stage or platen during printing facilitates solvent evaporation during the deposition process.

In some aspects, the platen or substrate temperature during printing may be maintained at a temperature of about 30 °C to about 80 °C, about 50 °C to about 70 °C, or about 40 °C to about 60 °C. In certain non-limiting exemplary embodiments, the platen temperature is about 50 °C. Maintaining the substrate at these temperatures promotes controlled solvent evaporation, which facilitates uniform polymer film formation and reduces defects such as pinholes or cracking. These temperatures can also improve adhesion of deposited materials by enhancing wetting and interfacial bonding without causing thermal degradation of sensitive components such as polymers or flexible substrates.

In some aspects, the aerosol jet printing nozzle size may be selected based on the desired printing resolution and specific material. Suitable nozzle sizes may include, for example, nozzles with nominal inner diameters of about 100 μm to about 500 μm. In some embodiments, the nozzle nominal inner diameter is about 350 μm to about 500 μm for UGI printing, or about 200 μm to about 500 μm for printing of electrically conductive materials. In certain non-limiting exemplary embodiments, the nozzle nominal inner diameter is about 410 μm for UGI printing and about 250 μm for sensor printing.

The disclosed printing methods enable one-step fabrication of both UGIs and functional bioelectronic devices using the same AMMP system. Taking advantage of the multi-material printing process, UGIs and functional devices can be printed all from a single nozzle by combining aerosolized ink streams including different polymers and electrically conductive materials, significantly improving fabrication efficiency. The unique capability for in situ aerosol-phase mixing and printing enables instantaneous tuning of material compositions.

### Embodiments

One embodiment described herein is a printed universal gradient interface for stretchable and strain-insensitive bioelectronics, the universal gradient interface comprising a multi-polymer gradient extending between a first polymer end and a second polymer end, the first polymer end comprising a first polymer content, the second polymer end comprising a second polymer content, and the multi-polymer gradient comprising a graded weight ratio of the first polymer content to the second polymer content of between 1:0 and 0:1 from the first polymer end to the second polymer end. In one aspect, each of the first polymer content and the second polymer content comprises a polyurethane polymer, a polyurethane dispersion (PUD) polymer, a thermoplastic polyurethane (TPU) polymer, a polynitrile polymer, a polyether polymer, a polycarbonate polymer, a polyalcohol polymer, a polyethylene polymer, a polyethylene oxide polymer, a polyethylene glycol polymer, a polypropylene polymer, a polyacrylate polymer, a polyester polymer, a polyvinyl polymer, a polyphosphazene polymer, a polysulfone polymer, a polysiloxane polymer, a polyamide polymer, a polylactic acid polymer, a polyacrylate polymer, a polyimide polymer, a fluoropolymer, or combinations thereof. In another aspect, the multi-polymer gradient comprises a polyurethane dispersion (PUD) gradient extending between a first PUD end and a second PUD end, the first PUD end comprising a first PUD content, the second PUD end comprising a second PUD content, and the multi-polymer gradient comprising a graded weight ratio of the first PUD content to the second PUD content of between 1:0 and 0:1 from the first PUD end to the second PUD end. In another aspect, each of the first PUD content and the second PUD content comprises one or more of a polyether-based PUD, a polyester-based PUD, or a polycarbonate-based PUD. In another aspect, the first PUD content comprises flexible polyester polyol segments and the second PUD content comprises stiff polyester polyol segments derived from methylene diphenyl diisocyanate (MDI). In another aspect, the first polymer content has a Young's modulus of about 0.05 MPa to about 80 MPa and the second polymer content has a Young's modulus of about 100 MPa to about 50 GPa. In another aspect, the multi-polymer gradient has a Young's modulus ranging from about 0.05 MPa at the first polymer end to about 50 GPa at the second polymer end. In another aspect, the universal gradient interface has a thickness of about 0.5 μm to about 2 mm. In another aspect, the second polymer end is configured to functionally connect to an electrically conductive material. In another aspect, the electrically conductive material comprises one or more of a metal, a semiconductor, a conductive polymer, or an electrode.

Another embodiment described herein is a printed bioelectronic device, comprising: a universal gradient interface comprising a multi-polymer gradient extending between a first polymer end and a second polymer end, the first polymer end comprising a first polymer content, the second polymer end comprising a second polymer content, and the multi-polymer gradient comprising a graded weight ratio of the first polymer content to the second polymer content of between 1:0 and 0:1 from the first polymer end to the second polymer end; and an electrically conductive material functionally connected to the second polymer end of the universal gradient interface. In one aspect, each of the first polymer content and the second polymer content comprises a polyurethane polymer, a polyurethane dispersion (PUD) polymer, a thermoplastic polyurethane (TPU) polymer, a polynitrile polymer, a polyether polymer, a polycarbonate polymer, a polyalcohol polymer, a polyethylene polymer, a polyethylene oxide polymer, a polyethylene glycol polymer, a polypropylene polymer, a polyacrylate polymer, a polyester polymer, a polyvinyl polymer, a polyphosphazene polymer, a polysulfone polymer, a polysiloxane polymer, a polyamide polymer, a polylactic acid polymer, a polyacrylate polymer, a polyimide polymer, a fluoropolymer, or combinations thereof. In another aspect, the multi-polymer gradient of the universal gradient interface comprises a polyurethane dispersion (PUD) gradient extending between a first PUD end and a second PUD end, the first PUD end comprising a first PUD content, the second PUD end comprising a second PUD content, and the multi-polymer gradient comprising a graded weight ratio of the first PUD content to the second PUD content of between 1:0 and 0:1 from the first PUD end to the second PUD end. In another aspect, the electrically conductive material comprises one or more of a metal, a semiconductor, a conductive polymer, or an electrode. In another aspect, the first polymer end of the universal gradient interface is configured to functionally connect to a biological surface. In another aspect, the biological surface comprises a mammalian tissue or organ. In another aspect, the mammalian tissue or organ comprises human skin or a human heart. In another aspect, the bioelectronic device is a wearable bioelectronic device. In another aspect, the bioelectronic device is a sensor comprising a temperature sensor, a photodetector, a pulse sensor, or an oximeter. In another aspect, the bioelectronic device exhibits improved strain insensitivity and resistivity changes under a high stretch ratio relative to conventional bioelectronic devices not having the universal gradient interface. In another aspect, the bioelectronic device withstands a uniaxial stretch ratio of up to about 180% without electrical failure. In another aspect, the bioelectronic device exhibits a relative resistance change of less than about 5% under a uniaxial stretch ratio of up to 100%.

Another embodiment described herein is a method of making a universal gradient interface for stretchable and strain-insensitive bioelectronics, the method comprising: mixing a first aerosolized ink stream comprising a first polymer and a second aerosolized ink stream comprising a second polymer using a carrier gas to form an aerosolized polymer mixture; and aerosol jet printing the aerosolized polymer mixture onto a substrate material using a sheath gas such that the first aerosolized ink stream comprising the first polymer is first deposited onto the substrate material to form a first polymer end comprising the first polymer; wherein flow rates of the first aerosolized ink stream and the second aerosolized ink stream are continuously adjusted to form a multi-polymer gradient extending between the first polymer end and a second polymer end comprising the second polymer, the multi-polymer gradient comprising a graded weight ratio of the first polymer to the second polymer of between 1:0 and 0:1 from the first polymer end to the second polymer end. In one aspect, the substrate material is treated with plasma to improve surface hydrophilicity prior to aerosol jet printing. In another aspect, the method further comprises drying the universal gradient interface to remove any solvent. In another aspect, the method further comprises aerosol jet printing a third aerosolized ink stream comprising an electrically conductive material onto the second polymer end, thereby functionally connecting the second polymer end to the electrically conductive material. In another aspect, the electrically conductive material comprises one or more of a metal, a semiconductor, a conductive polymer, or an electrode. In another aspect, aerosol jet printing comprises a sheath gas flow rate of about 50 sccm to about 70 sccm. In another aspect, aerosol jet printing comprises an aerosol jet print speed of about 1 mm/s to about 5 mm/s. In another aspect, each of the flow rates of the first aerosolized ink stream and the second aerosolized ink stream ranges from about 0 sccm to about 35 sccm. In another aspect, one or more of the first aerosolized ink stream and the second aerosolized ink stream further comprise one or more additives, solvents, or surfactants. In another aspect, the first polymer has a Young's modulus of about 0.05 MPa to about 80 MPa and the second polymer has a Young's modulus of about 100 MPa to about 50 GPa.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A printed universal gradient interface for stretchable and strain-insensitive bioelectronics, the universal gradient interface comprising a multi-polymer gradient extending between a first polymer end and a second polymer end, the first polymer end comprising a first polymer content, the second polymer end comprising a second polymer content, and the multi-polymer gradient comprising a graded weight ratio of the first polymer content to the second polymer content of between 1:0 and 0:1 from the first polymer end to the second polymer end.
Clause 2. The universal gradient interface of clause 1, wherein each of the first polymer content and the second polymer content comprises a polyurethane polymer, a polyurethane dispersion (PUD) polymer, a thermoplastic polyurethane (TPU) polymer, a polynitrile polymer, a polyether polymer, a polycarbonate polymer, a polyalcohol polymer, a polyethylene polymer, a polyethylene oxide polymer, a polyethylene glycol polymer, a polypropylene polymer, a polyacrylate polymer, a polyester polymer, a polyvinyl polymer, a polyphosphazene polymer, a polysulfone polymer, a polysiloxane polymer, a polyamide polymer, a polylactic acid polymer, a polyacrylate polymer, a polyimide polymer, a fluoropolymer, or combinations thereof.
Clause 3. The universal gradient interface of clause 1 or 2, wherein the multi-polymer gradient comprises a polyurethane dispersion (PUD) gradient extending between a first PUD end and a second PUD end, the first PUD end comprising a first PUD content, the second PUD end comprising a second PUD content, and the multi-polymer gradient comprising a graded weight ratio of the first PUD content to the second PUD content of between 1:0 and 0:1 from the first PUD end to the second PUD end.
Clause 4. The universal gradient interface of any one of clauses 1-3, wherein each of the first PUD content and the second PUD content comprises one or more of a polyether-based PUD, a polyester-based PUD, or a polycarbonate-based PUD.
Clause 5. The universal gradient interface of any one of clauses 1-4, wherein the first PUD content comprises flexible polyester polyol segments and the second PUD content comprises stiff polyester polyol segments derived from methylene diphenyl diisocyanate (MDI).
Clause 6. The universal gradient interface of any one of clauses 1-5, wherein the first polymer content has a Young's modulus of about 0.05 MPa to about 80 MPa and the second polymer content has a Young's modulus of about 100 MPa to about 50 GPa.
Clause 7. The universal gradient interface of any one of clauses 1-6, wherein the multi-polymer gradient has a Young's modulus ranging from about 0.05 MPa at the first polymer end to about 50 GPa at the second polymer end.
Clause 8. The universal gradient interface of any one of clauses 1-7, wherein the universal gradient interface has a thickness of about 0.5 μm to about 2 mm.
Clause 9. The universal gradient interface of any one of clauses 1-8, wherein the second polymer end is configured to functionally connect to an electrically conductive material.
Clause 10. The universal gradient interface of any one of clauses 1-9, wherein the electrically conductive material comprises one or more of a metal, a semiconductor, a conductive polymer, or an electrode.
Clause 11. A printed bioelectronic device, comprising:
   a universal gradient interface comprising a multi-polymer gradient extending between a first polymer end and a second polymer end, the first polymer end comprising a first polymer content, the second polymer end comprising a second polymer content, and the multi-polymer gradient comprising a graded weight ratio of the first polymer content to the second polymer content of between 1:0 and 0:1 from the first polymer end to the second polymer end; and
   an electrically conductive material functionally connected to the second polymer end of the universal gradient interface.
Clause 12. The bioelectronic device of clause 11, wherein each of the first polymer content and the second polymer content comprises a polyurethane polymer, a polyurethane dispersion (PUD) polymer, a thermoplastic polyurethane (TPU) polymer, a polynitrile polymer, a polyether polymer, a polycarbonate polymer, a polyalcohol polymer, a polyethylene polymer, a polyethylene oxide polymer, a polyethylene glycol polymer, a polypropylene polymer, a polyacrylate polymer, a polyester polymer, a polyvinyl polymer, a polyphosphazene polymer, a polysulfone polymer, a polysiloxane polymer, a polyamide polymer, a polylactic acid polymer, a polyacrylate polymer, a polyimide polymer, a fluoropolymer, or combinations thereof.
Clause 13. The bioelectronic device of clause 11 or 12, wherein the multi-polymer gradient of the universal gradient interface comprises a polyurethane dispersion (PUD) gradient extending between a first PUD end and a second PUD end, the first PUD end comprising a first PUD content, the second PUD end comprising a second PUD content, and the multi-polymer gradient comprising a graded weight ratio of the first PUD content to the second PUD content of between 1:0 and 0:1 from the first PUD end to the second PUD end.
Clause 14. The bioelectronic device of any one of clauses 11-13, wherein the electrically conductive material comprises one or more of a metal, a semiconductor, a conductive polymer, or an electrode.
Clause 15. The bioelectronic device of any one of clauses 11-14, wherein the first polymer end of the universal gradient interface is configured to functionally connect to a biological surface.
Clause 16. The bioelectronic device of any one of clauses 11-15, wherein the biological surface comprises a mammalian tissue or organ.
Clause 17. The bioelectronic device of any one of clauses 11-16, wherein the mammalian tissue or organ comprises human skin or a human heart.
Clause 18. The bioelectronic device of any one of clauses 11-17, wherein the bioelectronic device is a wearable bioelectronic device.
Clause 19. The bioelectronic device of any one of clauses 11-18, wherein the bioelectronic device is a sensor comprising a temperature sensor, a photodetector, a pulse sensor, or an oximeter.
Clause 20. The bioelectronic device of any one of clauses 11-19, wherein the bioelectronic device exhibits improved strain insensitivity and resistivity changes under a high stretch ratio relative to conventional bioelectronic devices not having the universal gradient interface.
Clause 21. The bioelectronic device of any one of clauses 11-20, wherein the bioelectronic device withstands a uniaxial stretch ratio of up to about 180% without electrical failure.
Clause 22. The bioelectronic device of any one of clauses 11-21, wherein the bioelectronic device exhibits a relative resistance change of less than about 5% under a uniaxial stretch ratio of up to 100%.
Clause 23. A method of making a universal gradient interface for stretchable and strain-insensitive bioelectronics, the method comprising:
   mixing a first aerosolized ink stream comprising a first polymer and a second aerosolized ink stream comprising a second polymer using a carrier gas to form an aerosolized polymer mixture; and
   aerosol jet printing the aerosolized polymer mixture onto a substrate material using a sheath gas such that the first aerosolized ink stream comprising the first polymer is first deposited onto the substrate material to form a first polymer end comprising the first polymer;
   wherein flow rates of the first aerosolized ink stream and the second aerosolized ink stream are continuously adjusted to form a multi-polymer gradient extending between the first polymer end and a second polymer end comprising the second polymer, the multi-polymer gradient comprising a graded weight ratio of the first polymer to the second polymer of between 1:0 and 0:1 from the first polymer end to the second polymer end.
Clause 24. The method of clause 23, wherein the substrate material is treated with plasma to improve surface hydrophilicity prior to aerosol jet printing.
Clause 25. The method of clause 23 or 24, further comprising drying the universal gradient interface to remove any solvent.
Clause 26. The method of any one of clauses 23-25, further comprising aerosol jet printing a third aerosolized ink stream comprising an electrically conductive material onto the second polymer end, thereby functionally connecting the second polymer end to the electrically conductive material.
Clause 27. The method of any one of clauses 23-26, wherein the electrically conductive material comprises one or more of a metal, a semiconductor, a conductive polymer, or an electrode.
Clause 28. The method of any one of clauses 23-27, wherein aerosol jet printing comprises a sheath gas flow rate of about 50 sccm to about 70 sccm.
Clause 29. The method of any one of clauses 23-28, wherein aerosol jet printing comprises an aerosol jet print speed of about 1 mm/s to about 5 mm/s.
Clause 30. The method of any one of clauses 23-29, wherein each of the flow rates of the first aerosolized ink stream and the second aerosolized ink stream ranges from about 0 sccm to about 35 sccm.
Clause 31. The method of any one of clauses 23-30, wherein one or more of the first aerosolized ink stream and the second aerosolized ink stream further comprise one or more additives, solvents, or surfactants.
Clause 32. The method of any one of clauses 23-31, wherein the first polymer has a Young's modulus of about 0.05 MPa to about 80 MPa and the second polymer has a Young's modulus of about 100 MPa to about 50 GPa.

### Examples

### General Ink Formulations

Commercially available gold ink (10 wt % in Xylene) was purchased from UT Dots. AgNWs (1 wt % in isopropanol) ink was purchased from Nanostructured & Amorphous Materials. PEDOT: PSS (0.8 wt % in water) were purchased from Sigma-Aldrich. Soft (Baymedix CD 102) and hard (U 9380) polyurethane dispersion (PUD) inks were obtained from Covestro and Alberdingk Boley, respectively. Depending on the ink type, a small amount of isopropyl alcohol (IPA) was often added as a defoamer to suppress foam formation during ultrasonic atomizing. To avoid aggregation and ensure uniform dispersion, inks were sonicated for 15 min (Fisher Scientific sonicate bath, 9.5 L). Typical ink composition is shown in Table 1. The concentration of nanomaterial was determined by the drying weight method.

TABLE 1
Typical Ink Formulation of Nanoparticle Inks

| | | | Mass | Surfactants/ |
|---|---|---|---|---|
| Materials | Solvent | Co-solvent | percentage | Additives |
| Soft PUD | Water | DMSO | 30% | EG |
| Hard PUD | Water | | 27.3% | EG |
| PEDOT: PSS | Water | | 0.8% | |
| Gold | Xylene | | 2% | |
| AgNWs | Water | | 1% | |
| MXene | Water | | 0.8% | |
| MoS₂ | IPA | | 0.6% | Terpinol |

For the preparation of the MoS₂ ink, a two-electrode electrochemical cell was used for electrochemical intercalation. A thin piece of cleaved MoS₂ crystal was clamped by a copper alligator clip as the cathode, and a graphite rod was placed as the anode respectively. Tetraheptylammonium bromide (THAB)/acetonitrile (5 mg mL⁻¹) served as the electrolyte. The electrochemical intercalation was conducted under a voltage of 7.5 V for 1 h. Afterward, the as-intercalated MoS₂ was rinsed with DMF and sonicated in PVP/DMF (0.2 mol L⁻¹, Mw=40,000) for 30 min. The dispersion was centrifuged and washed twice with IPA to remove excessive PVP. Then, the dispersion in IPA was centrifuged at 3000 rpm for 5 min, and the precipitates were discarded. The final dispersion of exfoliated MoS₂ ink in IPA was ready for further printing.

The synthesis of Ti₃C₂ Tx MXene ink involved selectively etching 1 g of Ti₃AlC₂ with an acidic solution of 9 mL deionized water, 3 mL hydrofluoric acid, and 18 mL hydrochloric acid. This mixture was stirred in a high-density polyethylene container on a heated stir plate for 24 h. Post-etching, the solution was centrifuged and washed to a neutral pH, followed by delamination using anhydrous lithium chloride. The resulting slurry was further washed, centrifuged, and concentrated into ink, which was then dried in a vacuum oven at 100 °C, resulting in a free-standing film. Prior to shipping, the ink is treated with argon and sealed in aluminum foil for overnight delivery.

### Fabrication of the Strain-Isolating Substrate and Sensors

The motion control graphical user interface (GUI) controls the printer's x, y, and z motion stages. Real-time position and velocity were tracked for x, y, and z during the targeted move and jogging operations. Both aerosol ink flow rates were actively controlled concerning stage position to achieve gradient and voxelated films. For all high-resolution printing, the inks were primed under sonication for 30 min and then atomized via ultrasonication before being transferred to the printhead. A sheath flow focused the aerosolized ink stream to achieve high printing resolution. Before printing, gradient or homogeneous substrates were treated with plasma to improve surface hydrophilicity. During the AMMP process, a heating stage evaporated the ink solvents to minimize undesired drying effects. Depending on the type of combinatorial materials, additional flash sintering was adopted to achieve the desired microstructures and properties.

All designs of substrates and sensors were printed using AMMP. First, the substrates were designed in AutoCAD, and then the printing paths were automatically generated. To measure the strain distribution, all specimens were prepared with a length of 31 mm, a width of 21 mm, and a gradient composition. The process parameters, including atomizer settings, ink qualities, carrier and sheath gas flow conditions, printer head as well as nozzle geometry, the distance between the nozzle tip and substrate, substrate temperature, etc., are summarized in Tables 2-3.

TABLE 2
Aerosol Jet Printing Parameters for Each Gradient Substrate Design

| | Non- | Linear | Z- | XY- | XYZ- |
|---|---|---|---|---|---|
| | gra- | gra- | gra- | gra- | gra- |
| Parameters | dient | dient | dient | dient | dient |
| Nozzle nominal I.D. (μm) | 410 | 410 | 410 | 410 | 410 |
| Sheath gas flow rate (sccm) | 60 | 60 | 60 | 60 | 60 |
| Soft PUD ink flow rate (sccm) | 30 | 10-30 | 10-30 | 10-30 | 10-30 |
| Hard PUD ink flow rate (sccm) | 30 | 17-30 | 17-30 | 17-30 | 17-30 |
| Platen temperature (°C) | 50 | 50 | 50 | 50 | 50 |
| Print speed (mm/s) | 2 | 2 | 2 | 2 | 2 |
| Ultrasonic atomizing | 43 | 43 | 43 | 43 | 43 |
| voltage (V) (soft) | | | | | |
| Ultrasonic atomizing | 43 | 43 | 43 | 43 | 43 |
| voltage (V) (hard) | | | | | |

TABLE 3
Aerosol Jet Printing Parameters for Each Sensor

| | | Temper- | | |
|---|---|---|---|---|
| | Strain | ature | Oximeter | Oximeter |
| Parameters | Sensor | Sensor | (channel) | (electrode) |
| Nozzle nominal I.D. (μm) | 250 | 250 | 410 | 250 |
| Sheath gas flow rate (sccm) | 80 | 80 | 60 | 80 |
| Ink flow rate (sccm) | 10 | 10 | 15 | 12 |
| Platen temperature (°C) | 50 | 50 | 50 | 50 |
| Print speed (mm/s) | 2 | 2 | 2 | 2 |
| Ultrasonic atomizing | 43 | 43 | 43 | 43 |
| voltage (V) | | | | |

### Digital Image Correlation (DIC)

The 2D DIC method was used to characterize the strain distribution and probe Young's modulus distribution of all 5 cases of printed substrates (1D non-gradient, 1D in-plane gradient, 1D out-of-plane gradient, 2D in-plane gradient, and 3D gradient). The force-displacement relation was recorded by uniaxial stretching of the specimen up to failure at a rate of 0.5% (of the initial length) per second via an Instron universal machine (model 5944) with a 50 N load cell. The specimen was mounted in a pair of pneumatic tensile grips, leaving a gauge length of 21 mm. To measure the strain distribution by the DIC method, an ink (Koh-I-Noor Rapidraw) was sprayed with an airbrush (Badger, no. 150) to generate high-quality speckle patterns on the specimen. A whiteboard was used as a background, and a white LED light was shone on the sample during testing to enhance the optical contrast. A Canon ESO 6D DSLR camera recorded changes in the speckle patterns with a Canon 100 mm F/2.8 L macro lens at roughly every 2% strain. The resolution of each image was ~15 μm per pixel, and data was recorded for every four-pixel length. Images were analyzed by Ncorr40, an open-source 2D DIC MATLAB software, to obtain the Eulerian strain distributions of the middle region of 11 mm (L)×11 mm (W). Based on the strain distribution, the modulus distribution was further calculated. See, e.g., Zeng et al., Nature 617:292-298 (2023).

### Finite Element Analysis (FEA)

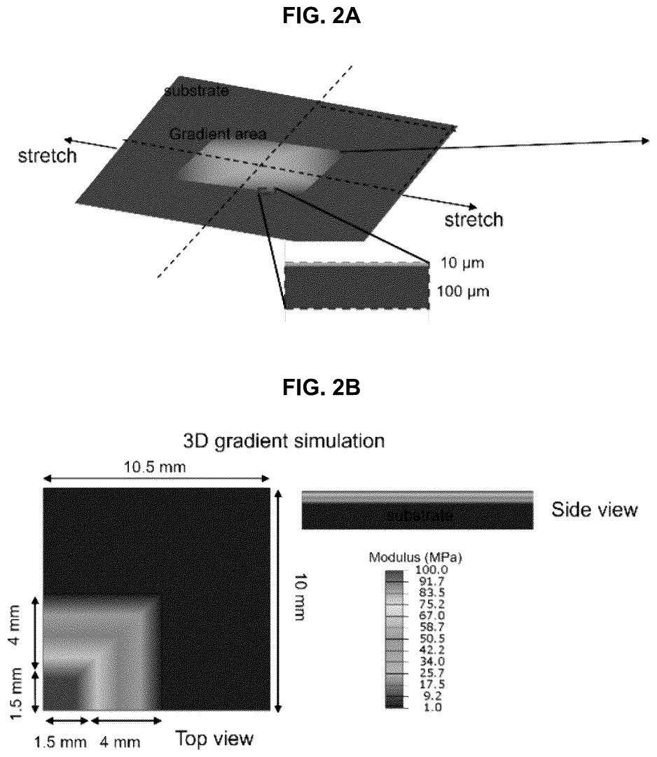

The design can be applied to substrates in wearable devices. It was assumed that the sample could be intimately attached to an arbitrary surface, such as the wrist, and stretched when rotating and bending. The sample can deform with the skin without failure. The commercial finite element software ABAQUS was utilized to simulate a gradient substrate's stress and strain distributions under uniaxial and biaxial tension. A 3D model with various modulus distributions designed along the x, y, and z directions was built. The thickness of the uniform soft substrate is 10 μm. On the top of the uniform substrate is a gradient square part with a length of 11 mm and a thickness of 1 μm (FIG. 2). The stress and strain contours in the x-y plane are shown in FIG. 2-3 and FIG. 12. By symmetry, only a quarter of the specimen is modeled separately with XSYMM and YSYMM boundary conditions at the left and bottom. An ABAQUS built-in hyperelastic model with the neo-Hookean form was assigned to the whole model. The shear moduli for the soft and hard PUD were set as 0.75 to 75 MPa, respectively, and the compressibility parameter to be D=0 to enforce incompressibility. The sample was stretched in the X direction up to 100% strain for the uniaxial tension case and simultaneously in the X and Y directions up to 100% strain for the biaxial tension case. Element type C₃D8H was applied and nonlinear static analysis.

### Nanoindentation

Nanoindentation experiments were conducted using a nanoindenter (Hysitron TriboIndenter, Bruker, Billerica, MA), applying a force of 70 UN to determine the elastic (Young's) modulus and hardness of the sample material.

### White Light Profilometer

Sample thickness was measured using a white light profilometer (Filmetrics, San Diego, CA) equipped with a 5× magnification objective, which employs chromatic aberration to accurately gauge the vertical displacement between the highest and lowest points of the sample's surface.

### Fluorescent Imaging

Two PUD inks were mixed with different fluorescent dyes and imaged to investigate the gradient composition of printed substrates. Specifically, a red color dye and a green color dye was added to the PUDs to visualize the composition change process better. The fluorescent dyes were purchased from GLO EFFEX with red (UVT-RD-1OZ) and green (UVT-GR-1OZ). The fluorescent images were captured with a Keyence BZ-X810 Microscope. RGB analyses quantified the intensity of the two colors on the printed gradient polyurethane films, where the RGB color profiles were obtained by using the ImageJ RGB-Profiler plugin (National Institutes of Health, USA).

### Resistance Analysis

A custom-built test setup was used to measure the resistance of electronics under different strains. The straining mechanism consisted of a stepper motor for driving the timing belt, a microcontroller unit to control the precise movement of the timing belt, and a base connected to the timing belt that holds the tested substrate to be stretched. The resistance of the strain gauge is measured using the DAQ system and LabVIEW. The data was continuously monitored and recorded on a computer.

### Flash Sintering

Flash sintering was performed using a Sinteron 2100 system (Xenon Corp., USA) with a 107 mm xenon spiral lamp. The S-2100 system was configured for maximum pulse durations of 3 ms, with the sintering carried out in an ambient environment. The S-2100 produced pulse energy (single) ranging from 30 to 2850 J. The detailed flash sintering process are described in Saeidi-Javash et al., Energy. Environ. Sci. 15:5093 (2022).

### Photodetector Characterization

Photoresponse measurements were conducted using a Keithley 2636A source meter under ambient conditions. A 405 nm laser (Crystal Laser) served as the light source, with power adjustments made via the diode current. Calibration was achieved using an energy meter (PM100D, Thor Labs). Spectrally resolved photocurrents were recorded across wavelengths from 360 to 800 nm at a constant power of 2.5 mW using a supercontinuum broadband laser (SuperK FIANIUM-NKT photonics) and bandwidth tunable filter (SuperK VARIA-NKT photonics). Response times were determined by dynamically modulating the laser with a shutter to control the periods of light exposure. The photocurrent mapping measurement is described in Zeng et al., Adv. Mater. 32 (39): 2003081 (2020). The driving voltage of the red and green LEDs was set at 3 V. At the same time, the oxygen saturation was measured by a commercial pulse oximeter (CMS50NA, Contact Medical Systems Co., Ltd.). The oxygen saturation (SpO₂) can be expressed as a function of the transmitted light ratio (R_{OS}) of green and red LEDs. The green and red PPG signal amplitudes were calculated and then calculated the R_{OS}.

An informed written consent from all human participants was obtained prior to the research.

### X-Y-Plane Printing Resolution

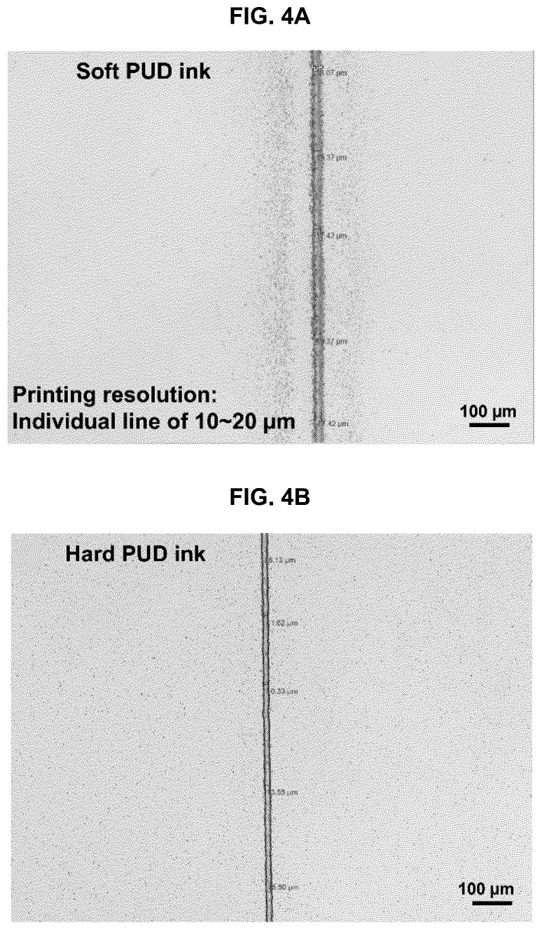

The aerosol-based multi-materials printing (AMMP) approach achieves a high resolution of approximately 20 μm in the X-Y-plane. The single filaments printed through soft PUD and hard PUD inks are shown in FIG. 4A-B.

### Z-Plane Printing Resolution

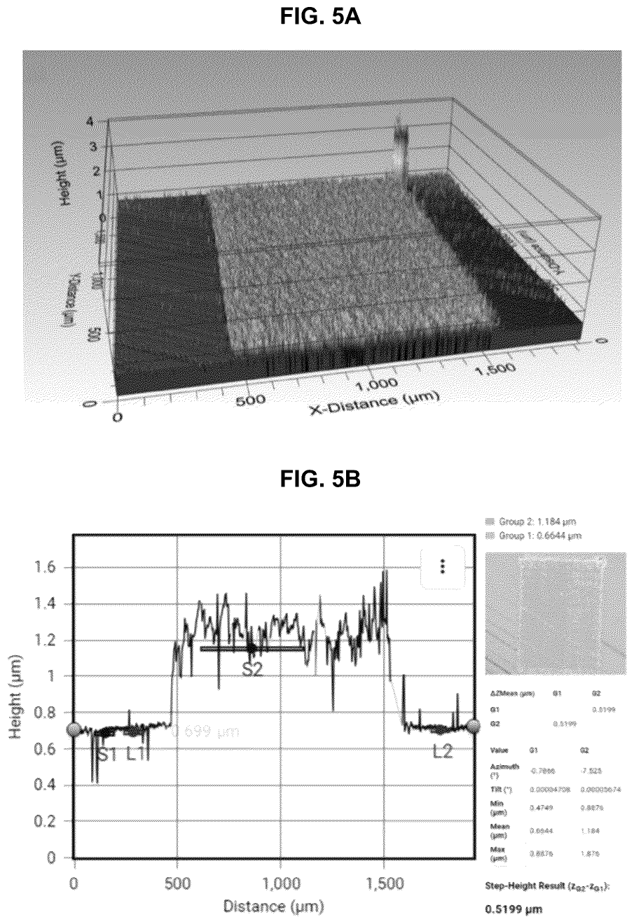

FIG. 5A shows the 3D scan of the printed 1-layer PUD film by using a white light profilometer (Filmetrics, Profilm3D). White light interferometry technology is used by this kind of profilometer to provide quantitative surface topological information. It is a nondestructive, non-contact type of measurement method offering good technology to measure film thickness. The film thickness shown in FIG. 5B demonstrates the deposition resolution along the Z-direction as small as around 500 nm.

### Design of Each Gradient Pattern

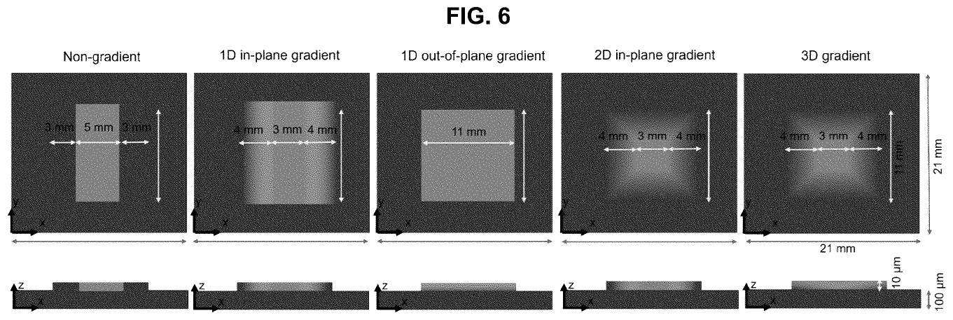

FIG. 6 illustrates the dimensions of various Universal Gradient Interface (UGI) designs. The non-gradient design, used as a reference, features an abrupt modulus transition along the X-direction. The 1D in-plane gradient design exhibits a gradual modulus change in the X-direction. Conversely, the 1D out-of-plane gradient design transitions modulus changes along the Z-direction. The 2D in-plane gradient design modifies modulus progressively in both the X and Y directions. Lastly, the 3D gradient design systematically alters the modulus across all three dimensions: X, Y, and Z.

### Non-Gradient and 1D Out-of-Plane UGI Properties

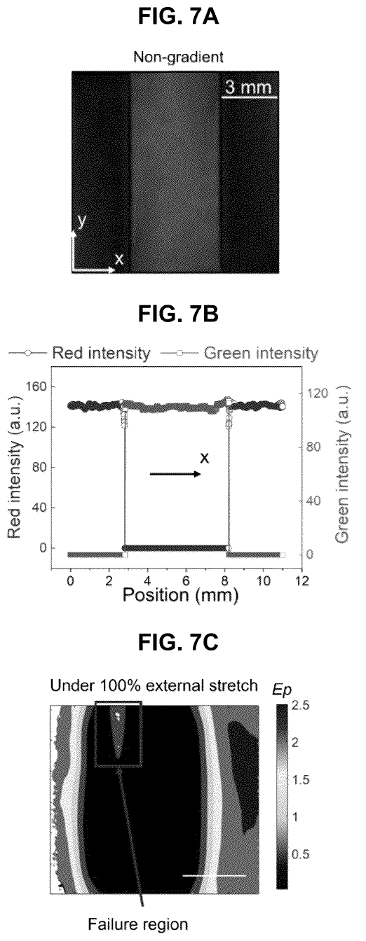

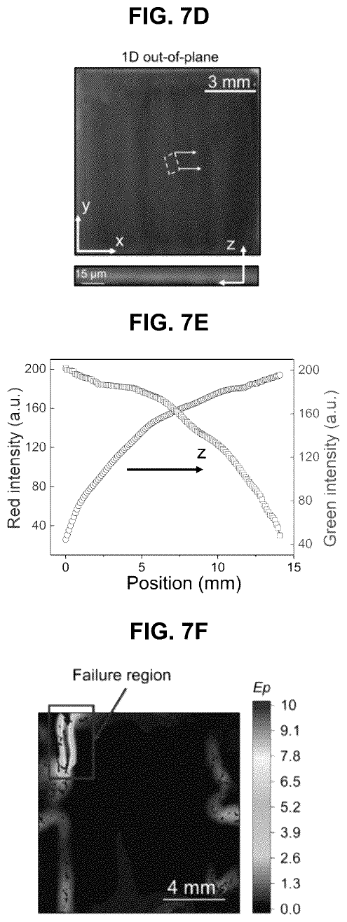

FIGS. 7A and 7D show the printed non-gradient and 1D out-of-plane interface patterns which are used for comparison purposes. FIGS. 7B and 7E show the site-specific component ratio of the non-gradient and 1D out-of-plane gradient design. The top surface principal Lagrange strain map obtained from DIC for non-gradient and 1D out-of-plane gradient samples under 100% global uniaxial external stretch (FIGS. 7C and 7F) illustrates the non-gradient and 1D out-of-plane gradient samples already break at the soft-stiff interfaces.

### DIC Figures for Each Design at Crack

FIG. 8 shows the top surface maximum principal logarithmic strain map obtained from DIC for each design when a crack occurs.

### Finite Element Analysis (FEA) Results Under Uniaxial Tension

FIG. 9 illustrates the maximum principal stress and logarithmic strain distributions obtained from FEA under 100% uniaxial tension for each design. In non-gradient designs, the abrupt modulus transition at soft-stiff interfaces leads to significant strain mismatches and elevated stress levels, particularly at the corners. Similarly, in 1D out-of-plane designs, stress and strain concentrations occur at the corners of the stiff region due to the absence of gradient transitions along the stretch direction. In contrast, UGI designs (1D in-plane, 2D in-plane, and 3D gradients) effectively mitigate stress and strain concentrations by providing smoother modulus transitions. These transitions reduce localized stress, allowing a broader region to distribute it more evenly. Additionally, the gradient designs relocate high-stress regions to the stiff (hard PU) areas, significantly enhancing global failure resistance. The strain progressively increases along the gradient as softer regions deform more under the same external loads.

Among these, the 3D gradient design achieved the smoothest strain distribution, further optimizing stress distribution and minimizing strain concentrations.

### Stretching Process for Each Design

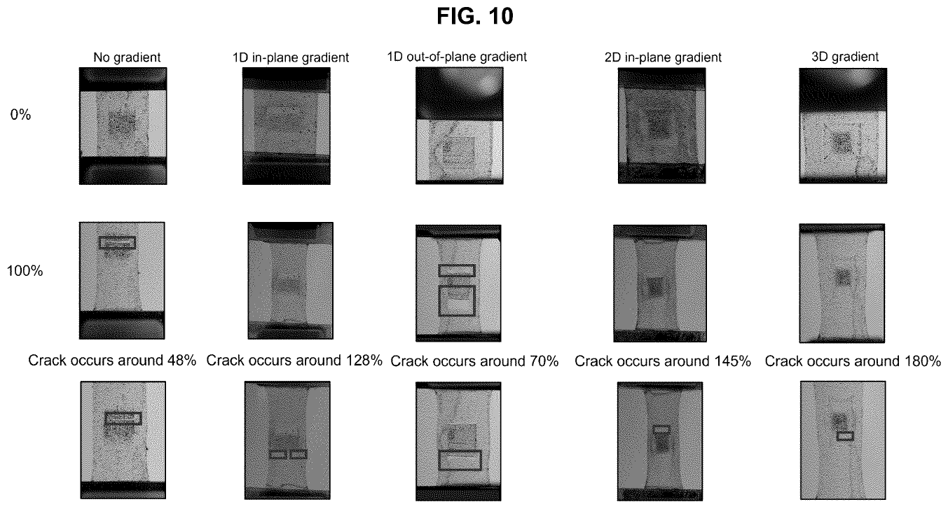

FIG. 10 shows the optical images of the stretching process of 0-100% uniaxial stretch ratio and the images of cracks occurring in each UGI.

### Tensile Test for Non-Gradient and 3D UGI Design

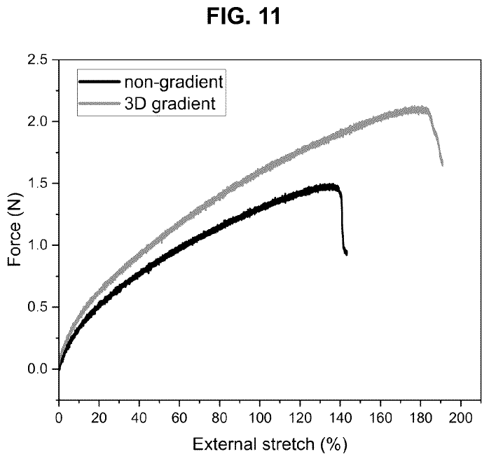

FIG. 11 shows the tensile test for non-gradient and 3D UGI design.

### Simulation for Each Design for Uniaxial Stretch Ratio of 100%

FIG. 2 shows the simulation results of maximum principal strain and principal maximum stress under a uniaxial stretch ratio of 100% for each UGI. FIG. 2A-B show the schematic and dimensions of the simulation process. By symmetry, only a quarter of the specimen was modeled with XSYMM and YSYMM boundary conditions at the left and bottom separately. FIG. 2C (top) illustrates the maximum principal logarithmic strain distribution of each design. FIG. 2C (bottom) shows the principal maximum stress distribution of each design.

### Simulation for Each Design for Bi-Axial Stretch Ratio of 100%

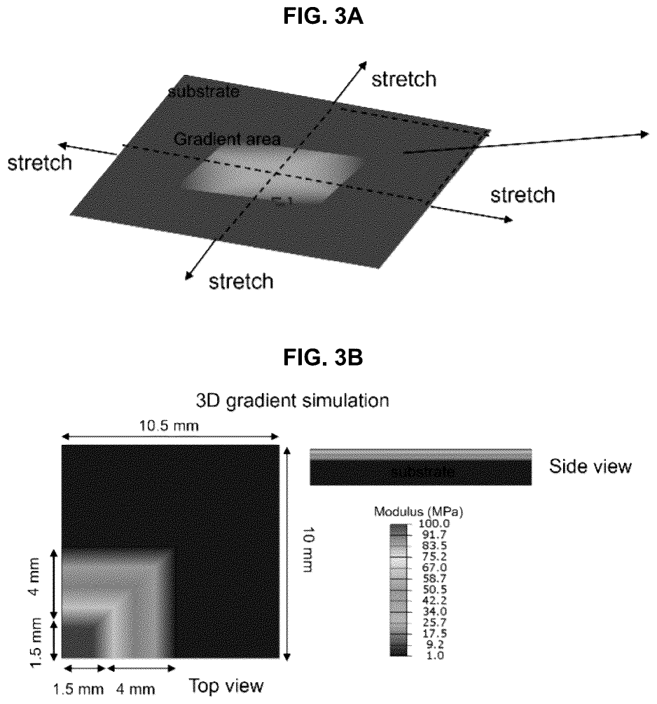

FIG. 3 shows simulations for each design for bi-axial stretch ratio of 100%. FIG. 3A shows a schematic of the simulation setup. FIG. 3B shows simulation dimensions with 3D in-plane gradient design as an example. FIG. 3C (top) shows principal logarithmic strain distribution of each design and FIG. 3C (bottom) shows maximum principal stress distribution of each design.

### Flash Sintering of Printed Au Sensor

Flash sintering is one type of photonic sintering process where white flashlight energy is utilized to create local heating, resulting in the evaporation of solvents, integration of the nanoparticles being sintered, increasing the electrical and thermal conductivity, reducing porosity, etc. In this study, a Sinteron 2100 (Xenon Corporation, USA) integrated with a 107 mm Xenon spiral flash lamp was used. The xenon flash lamp generates white light by converting the electrical energy into light energy. The short-pulsed white light from the xenon lamp covers a small part of UV light (380 nm), infrared light (950 nm), and the entire length of visible light. Due to the surface plasmon resonance effect of the metal nanoparticles in the range of the visible light spectrum, when flash sinters metal nanoparticles, they absorb the light energy, resulting in local heating and completing the sintering process. As the whole process is completed in a few seconds, the flash sintering is faster than other sintering processes, such as thermal sintering, laser sintering, etc. Targeted and localized sintering is also possible by flash sintering.

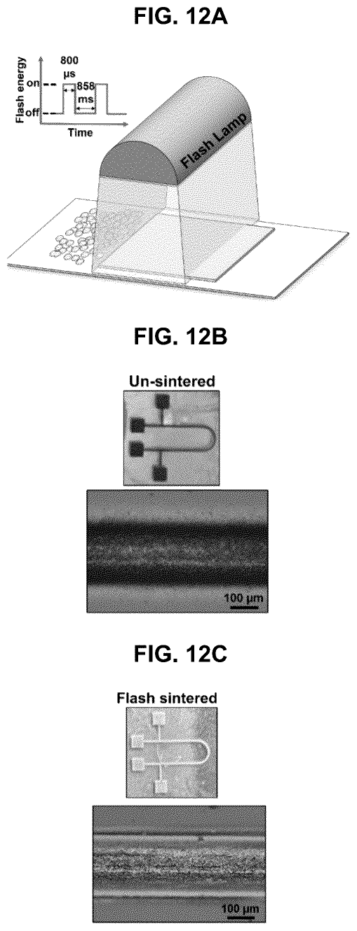

The printed gold nanoparticles were sintered in air at 2 kV flash lamp voltage, 800 μs flashlight duration, and 3 consecutive pulses with a pulse delay of 858 ms, as shown in FIG. 12A. As the optimized sintering time is less than 2 s and the PUD substrate only absorbs the UV light of the xenon flash lamp, metal nanoparticles can be sintered on a PUD substrate without potentially damaging it. The comparison of the printed gold filaments before and after flash sintering are illustrated in FIG. 12B-C.

### Stability Curve of the Printed MXene-Based Sensor

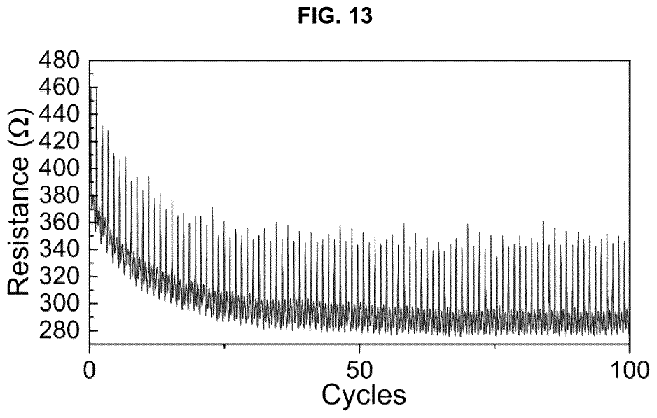

FIG. 13 shows the stability curve of the printed MXene-based sensor on soft PUD substrate is under 20% uniaxial strain for 100 cycles.

### 3D UGI Cycling Test

To validate the mechanical repeatability of the UGI and its ability to maintain stable electrical properties in integrated devices, a 1,000-cycle tensile test was conducted at a 50% stretch ratio using a gold-based sensor on a 3D gradient UGI (FIG. 14A). The resistance change of the sensor was recorded, revealing a low and repeatable relative resistance change (RRC) (ΔR/R0) of less than 1.6% throughout the cycles (FIG. 14B). These results highlight the durability and resilience of the 3D UGI under cyclic loading conditions.

### Au-Based Temperature Sensor

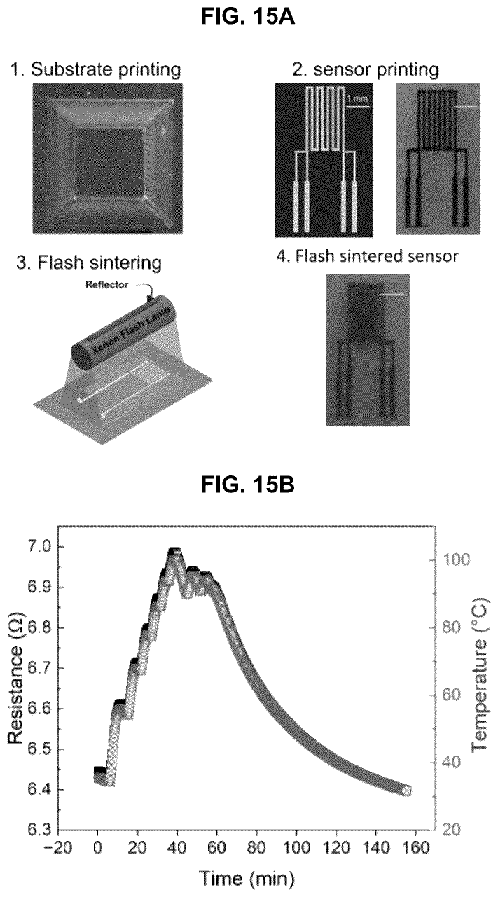

Here the detailed design and fabrication process of the stretchable temperature sensor are illustrated in FIG. 15A. FIG. 15B shows the resistance change of the temperature sensor accurately following the changed temperature, which demonstrates the accuracy of the temperature sensor.

### Humidity Test

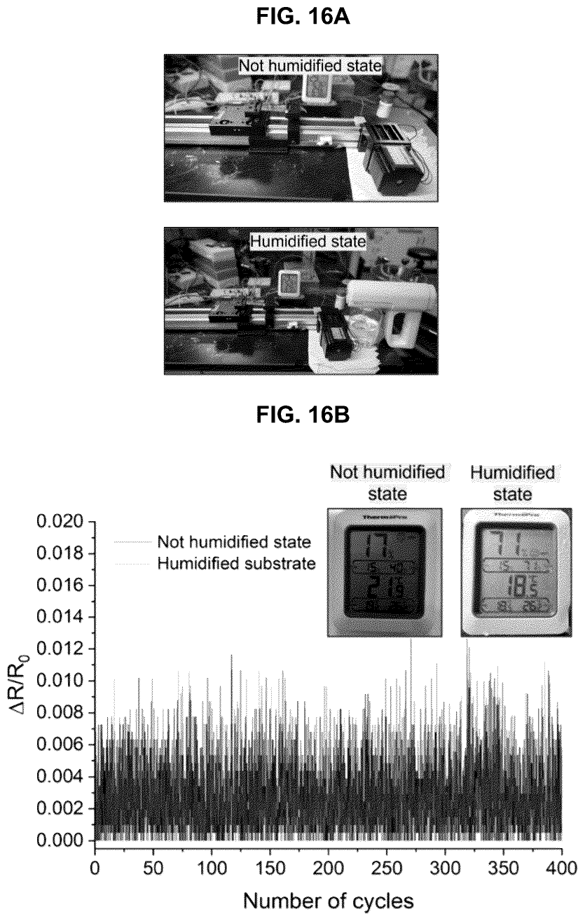

To further evaluate the UGI's performance under varying environmental conditions, its robustness was tested at different humidity levels. A humidifier was used to control the environmental humidity (FIG. 16A). The sensor printed on the 3D gradient UGI showed consistent performance with minimal RRC change (<1.3%) during stretching (50% stretch ratio) under different humidity conditions, confirming the platform's reliability (FIG. 16B).

### Substrate Transparency Analysis

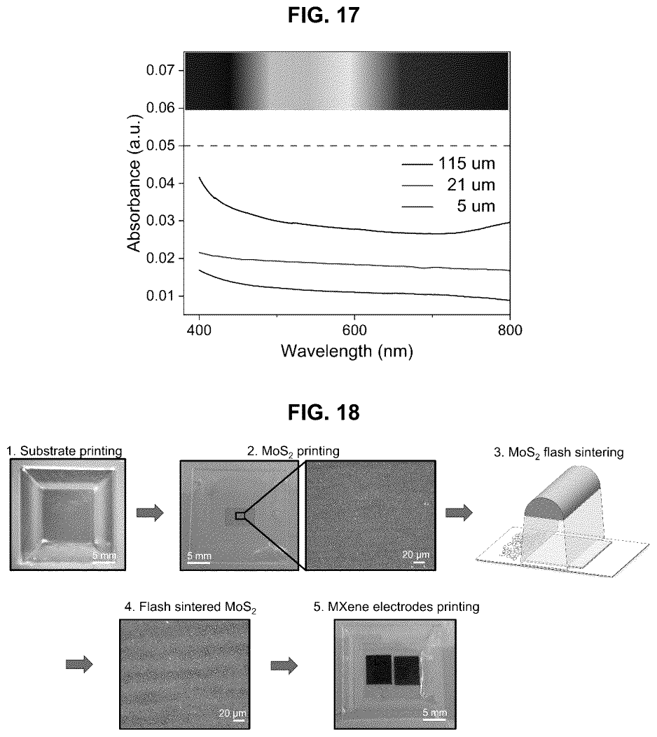

Here the transparency of PUD substrate under different wavelengths is illustrated in FIG. 17. The high transparency confirms its capability for optical sensor applications.

### Optical Sensor Fabrication Process

The MoS₂-based optical sensor or photodetector fabrication process is illustrated in FIG. 18. First, the MoS₂ was printed on a gradient substrate. Then, the MoS₂ layer was sintered in air at 2.4 kV flash lamp voltage, 800 μs flashlight duration, and 2 consecutive pulses with a pulse delay of 1360 ms through flashlight sintering. MXene was then printed on MoS₂ as electrodes.

### Responsivity of Printed Optical Sensor

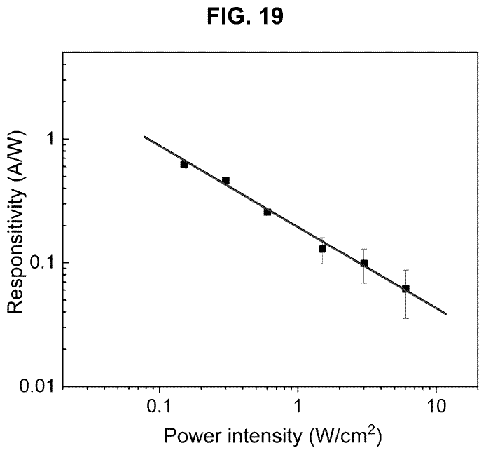

For measuring the photodetector responsivity, it can be assumed that the intensity is uniform throughout the device channel since the laser spot size is substantially larger than the channel and covers the entire device. The total power was determined by multiplying the channel area by the intensity at the set laser current. The effective channel area is approximately 300 μm×300 μm, and the input 405 nm laser has a diameter of 460 μm. Thus, responsivity can be calculated by R=I_{pc}/P, where I_{pc} is the photocurrent and P is the laser input power. The high responsivity observed is primarily due to the applied voltage across the photodetector, enhancing the electric field, which in turn affects charge carrier dynamics. This applied voltage improves photocurrent generation by decreasing carrier recombination and boosting light-to-electricity conversion efficiency. In MoS₂ photoresistors, which operate based on the photoconductive principle, such voltage application not only increases conductivity with light exposure but also facilitates charge separation, further amplifying the photocurrent and thereby enhancing responsivity. See FIG. 19.

Response Time and responsivity Relationship of Printed Optical Sensor

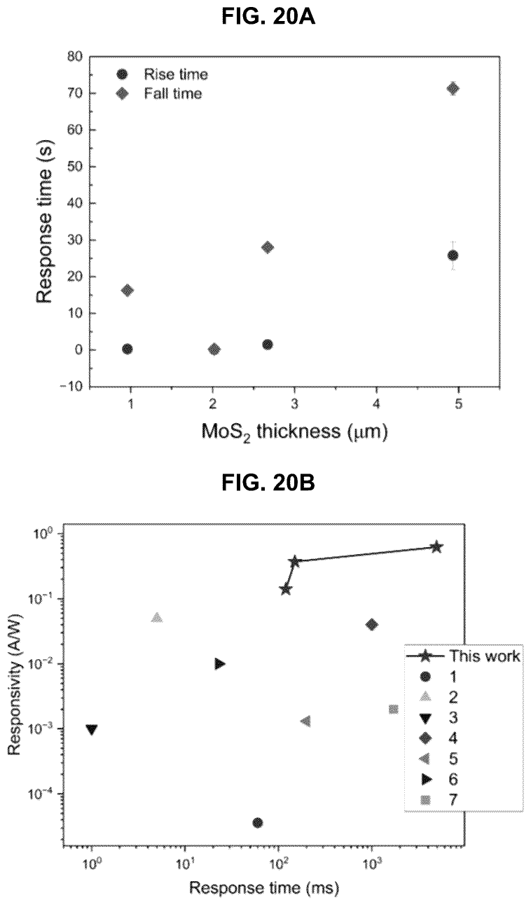

FIG. 20A shows the relationship between the printed MoS₂ thickness and response time. A thinner layer of MoS₂ in photodetectors generally leads to a faster response primarily due to the reduced volume of material through which charge carriers need to move. While too thinner MoS₂ layers might not absorb light as effectively due to their reduced thickness, which can decrease the generation rate of electron-hole pairs. This reduced generation rate means fewer carriers are available to contribute to the photocurrent, potentially increasing the time it takes to reach a detectable signal level. FIG. 20B shows the comparison of responsivity and response time in the devices in this work with previously reported all-printed photodetectors in the visible light range.

### Setup for Optical Sensor Array Application

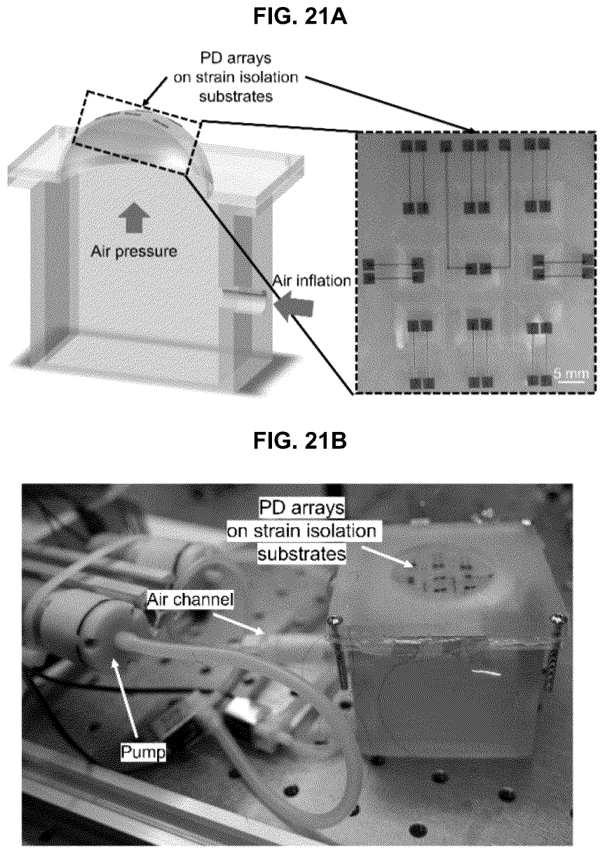

FIG. 21A-B show a schematic illustration of the cross-sectional view of the pressure-tunable cylindrical cavity and an optical image of the pressure-tunable cylindrical cavity connected with a mini pump.

### Simulation of Curved Surface Deformation

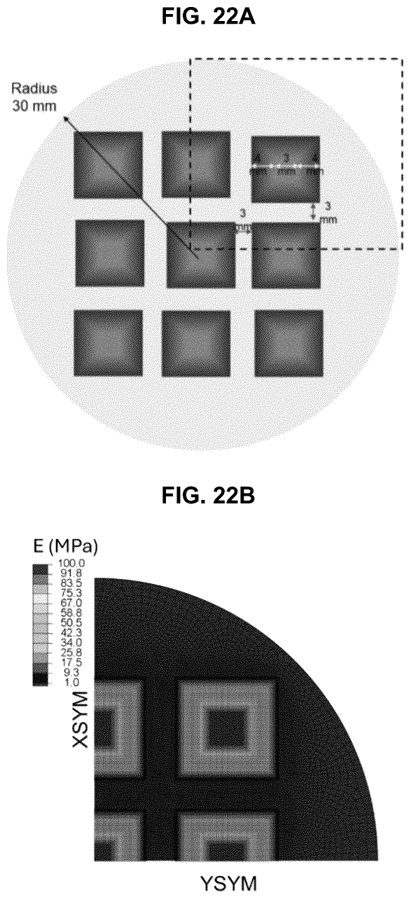

FIG. 22A illustrates the dimensions of the surface being simulated. Owing to symmetry, only one-quarter of the specimen is modeled, as depicted in FIG. 22B. FIG. 22C presents the simulated results of the strain and stress distributions.

### 3D Photodetector Array Location Coding and Photocurrent

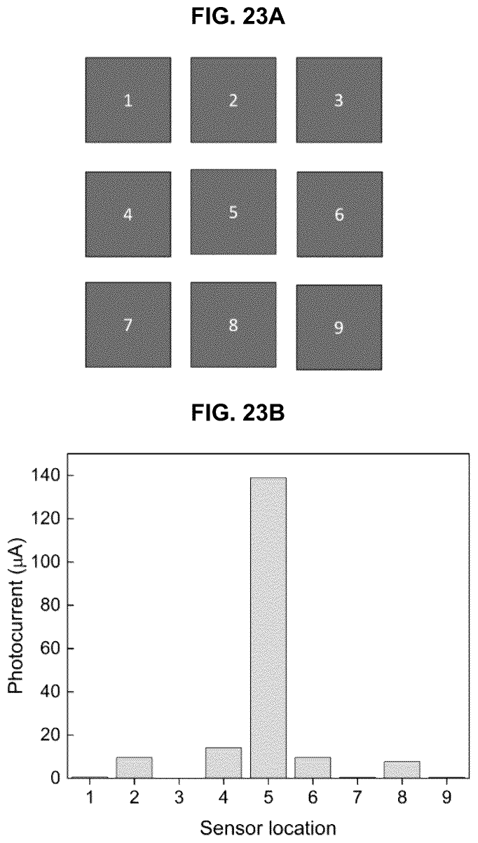

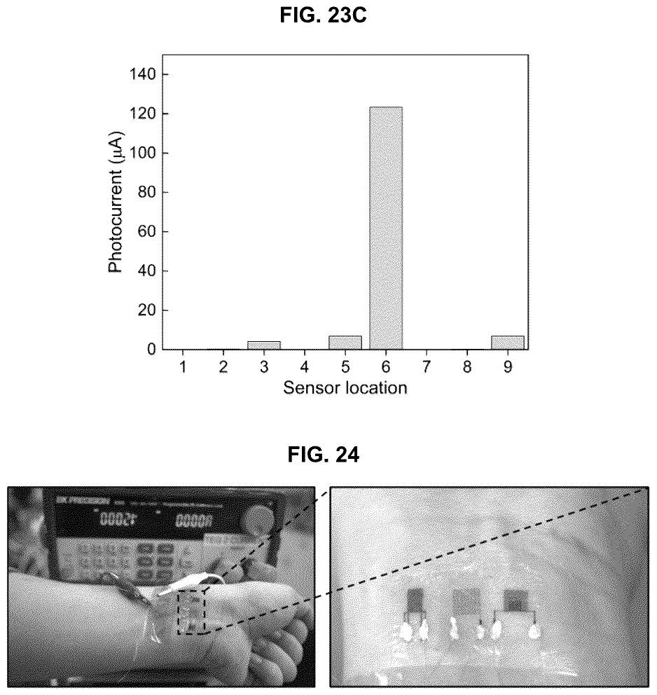

FIG. 23A-C show a 3D photodetector array location coding under normally-incident illumination and under obliquely incident light.

### Electrical Connection of the Demonstrators

FIG. 24 illustrates the connection setup for the skin-mounted sensors to the external measurement equipment during the experiments. Flexible conductive leads (36 AWG tinned copper wires) and adhesive electrodes (silver conductive paint) were utilized to ensure reliable and robust measurements while prioritizing user comfort. To secure the system, a thin layer of transparent tattoo paper was applied over the device, sealing it completely.

### Oximeter Calculation

Blood oxygen saturation (SO₂) is quantified according to Equation 1. Here, C_{HbO2} and C_{Hb} are the concentrations of oxy-hemoglobin and deoxy-hemoglobin, respectively.

$${SO}_{2} = \frac{{C}_{{HbO}_{2}}}{{C}_{{HbO}_{2}} + {C}_{Hb}} \qquad ( 1 )$$

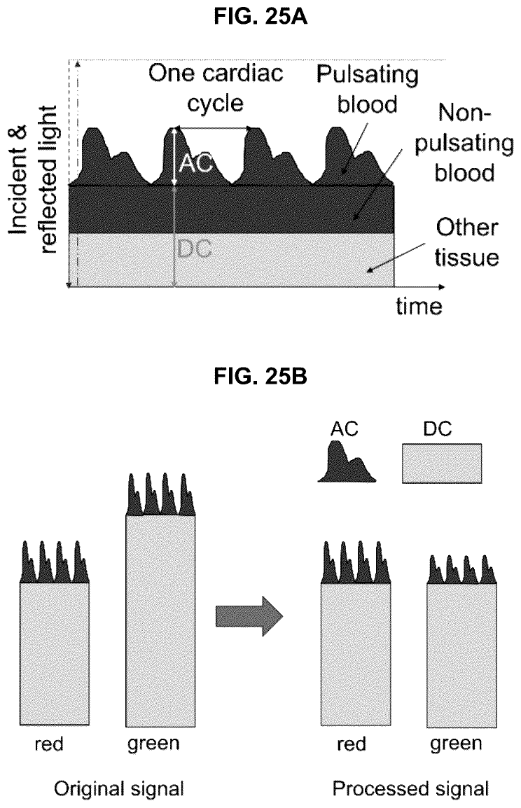

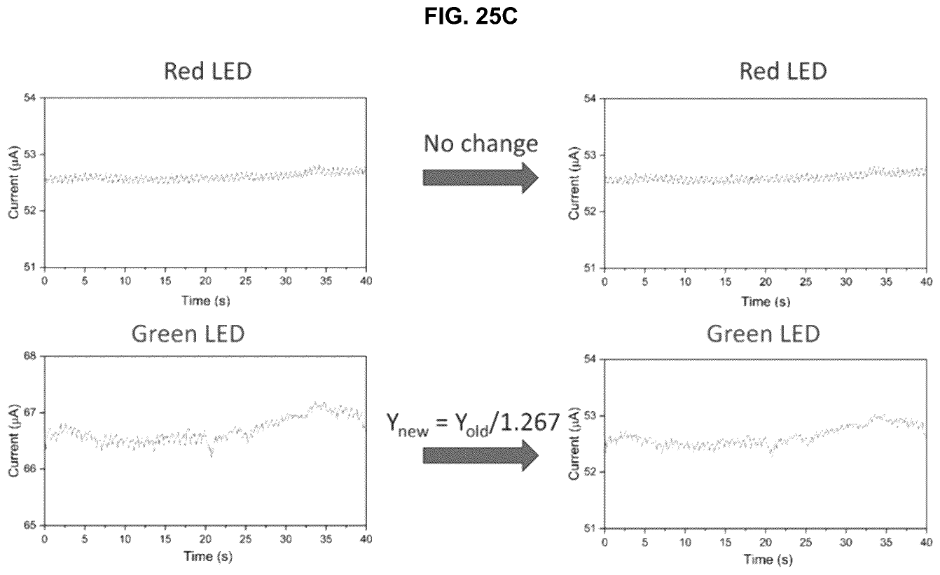

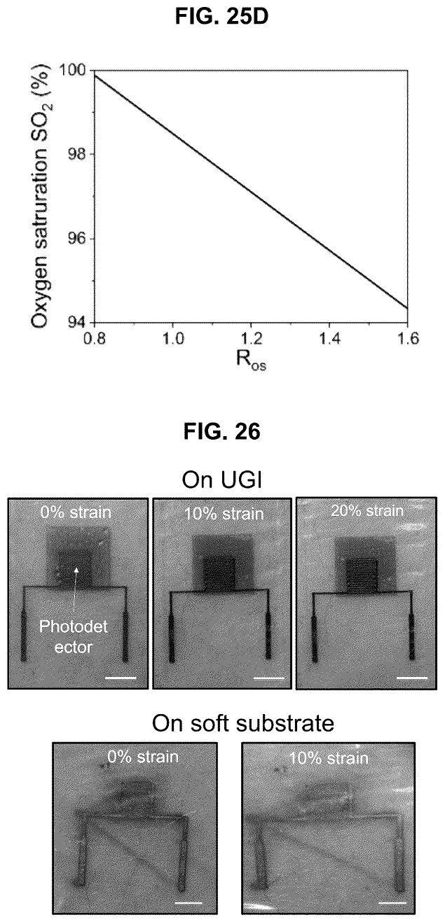

In this context, the characteristic value “Rₒₛ” is used, the ratio of signal from red (Ard) and green (Agr) light according to Beer-Lambert's law (shown in Equation 2) to represent the blood oxygen saturation (SO₂):

$${R}_{OS} = \frac{{A}_{rd}}{{A}_{gr}} \approx \frac{{AC}_{rd}}{{DC}_{rd}} / \frac{{AC}_{gr}}{{DC}_{gr}} \qquad ( 2 )$$

where AC_{rd} and DC_{rd}, along with AC_{gr} and DC_{gr}, represent signals derived from red (650 nm) and green (530 nm) light PPG waveforms, respectively. As demonstrated in FIG. 25A, these signals can be categorized into DC and AC components when converting light signals to electrical signals. The DC component is relatively constant across non-arterial tissues like muscles, bones, veins, and connective tissues, provided there is minimal movement at the measurement site. In contrast, the AC component varies due to dynamic changes in arterial light absorption, capturing fluctuations in arterial blood volume linked to cardiac cycles, specifically the phases of diastole and systole.

Direct comparison of red and green light signals may be unreliable due to variations in their light sources. The disparity in the DC component stems from the differing absorption rates of red and green light by skin tissue. To ensure equitable analysis of PPG signals from both light sources, the DC components are typically normalized through proportional calculations, as depicted in FIG. 25B-C. This normalization facilitates straightforward calculation of the R_{OS} value:

$${R}_{OS} \approx \frac{{AC}_{rd}}{{AC}_{gr}} \qquad ( 3 )$$

For illustration, the measurement is made during a breath hold process, where SO2 levels affect the oximeter readings. At an SO₂ of 99%, the AC_{rd} is 0.135 μA, and AC_{gr} is 0.150 μA, yielding a R_{OS} value of 0.9. When SO₂ drops to 96%, AC_{rd} increases to 0.178 μA while AC_{gr} decreases to 0.134 μA, resulting in a R_{OS} of 1.328. This demonstrates a significant rise in R_{OS} correlating with decreases in SO₂ during breath hold process. Consequently, the oximeter effectively measures and interprets real-time, non-invasive oxygen saturation, facilitating ongoing, discreet surveillance of physiological changes for early anomaly detection and disease prevention.

According to the Beer-Lambert Law SO₂ is determined by Equation S4, which directly correlates SO₂ with the R_{OS} value. The terms ε_{rd,Hb} and ε_{gr,Hb} represent the molar absorptivities of deoxy-hemoglobin at the red (λ=626 nm) and green (λ=530 nm) wavelengths, respectively. In a similar vein, ε_{rd,HbO2} and ε_{gr,HbO2} denote the molar absorptivities of oxy-hemoglobin at these same red and green wavelengths, respectively.

$${SO}_{2} ( {R}_{OS} ) = \frac{( {ε}_{rd , Hb} - {ε}_{grd , Hb} ) {R}_{OS}}{( {ε}_{rd , Hb} - {ε}_{rd , {Hb}_{{O}_{2}}} ) + ( {ε}_{gr , Hb} - {ε}_{gr , {Hb}_{{O}_{2}}} ) {R}_{OS}} \qquad ( 4 )$$

To address the limitations of the Beer-Lambert Law, empirical corrections are necessary. By aligning the collected Rₒₛ values with the SO2 values from a commercial sensor, a calibration curve is presented in FIG. 25D.

### On Skin Performance

FIG. 26 shows an oximeter printed on UGI and soft substrate before and after on-skin uniaxial stretching. Scale bar, 2.5 mm

### Universal Gradient Interfaces (UGIs) Using AMMP

AMMP allows to directly print the UGI with gradient modulus with submicron spatial resolution. The AMMP approach starts by atomizing two (or multiple) inks into aerosols that consist of microscale droplets. These aerosols are then mixed in situ in a single nozzle and focused using a co-flowing sheath gas prior to deposition (FIG. 1A). The AMMP enables precise control over local material composition by dynamically modulating the mixing ratio of different aerosolized inks. Taking advantage of the multi-material printing process, the UGI and functional devices can be printed all in a single nozzle, significantly improving fabrication efficiency.

In this work, the UGI was printed by mixing two polyurethane dispersions (PUDs) with drastically different moduli. The soft PUD contains flexible polyester polyol segments with low cross-linking, resulting in a lower modulus and higher elongation at break. Conversely, the stiff PUD is composed of hard segments derived from methylene diphenyl diisocyanate (MDI), leading to a higher modulus and lower elongation at break. This combination allows the elastic (Young's) modulus of the gradient material system to span two orders of magnitude, ranging from 1 MPa (soft PUD) to over 100 MPa (stiff PUD) (FIG. 1B). Upon stretching the UGI, the deformation concentrates in low modulus regions (red color) (FIG. 1B), which makes the high modulus region (green color) a strain-isolation region, on which printing the functional electronics is beneficial. The AMMP approach achieves a high resolution of ≈500 nm along the deposition thickness (Z-direction) and 20 μm in the X-Y plane (FIG. 4-5). For demonstration, a 3D gradient UGI is displayed where digital image correlation (DIC) reveals a 2.5% maximum principal Lagrange strain in stiff areas, while the whole 3D gradient UGI is under 100% equi-biaxial stretch, showcasing its superior strain isolation capability (FIG. 1B). This method effectively bridges the gap between soft tissues and a wide variety of stiff materials commonly used in electronic/sensing devices (FIG. 1C).

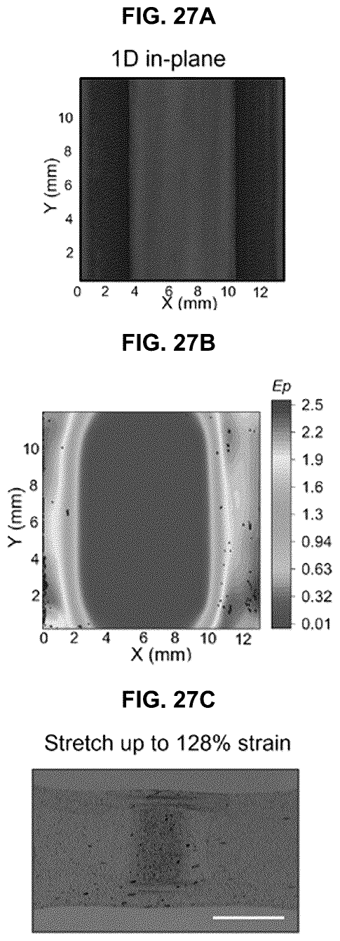

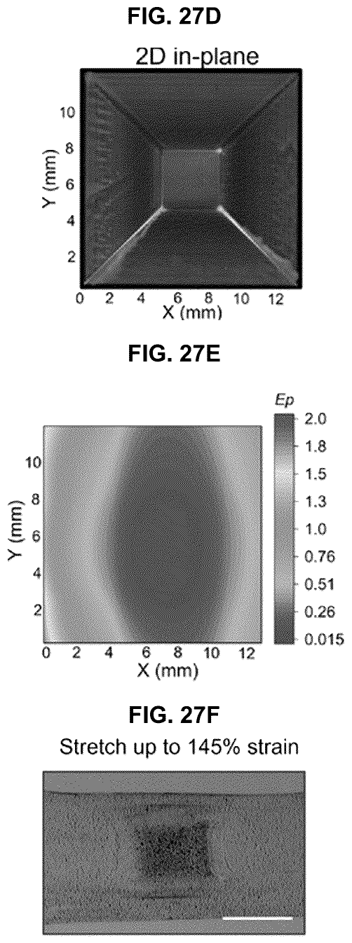

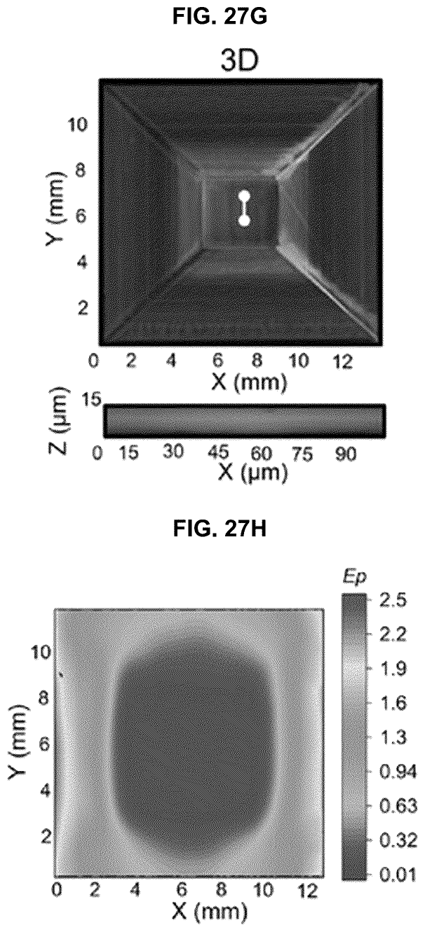

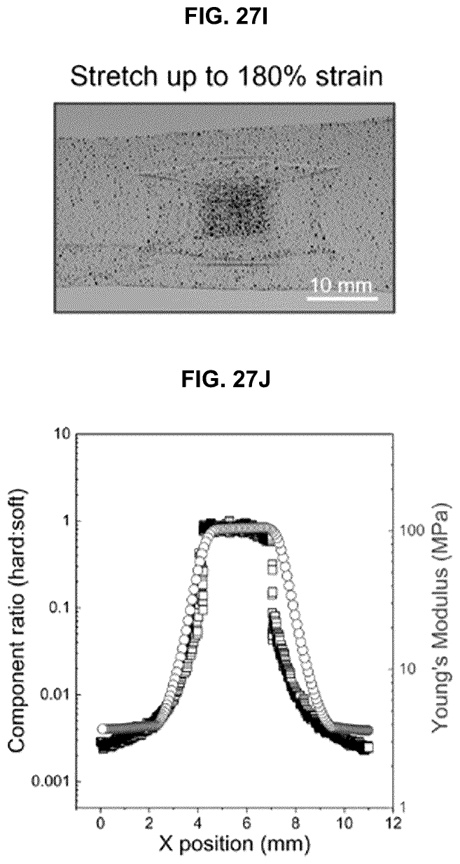

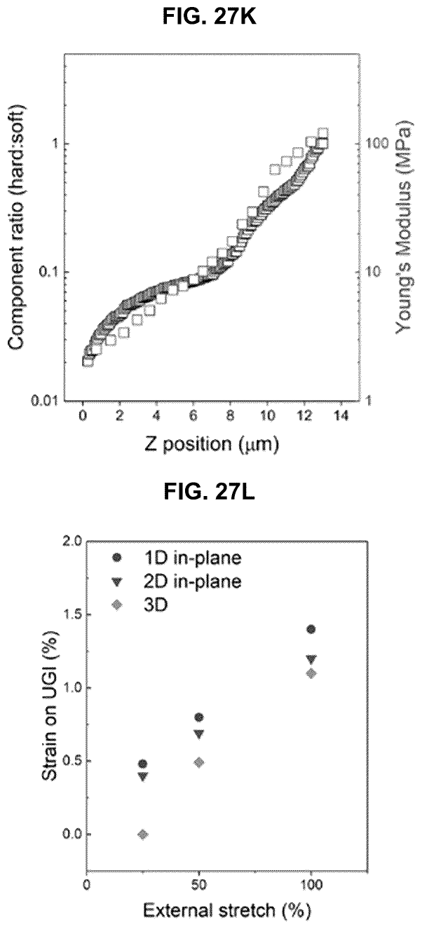

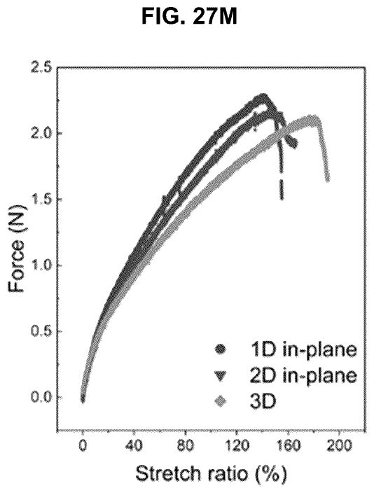

To validate the strain-isolation performance of the method, UGIs were first printed to assess the effect of stiffness gradients on strain and stress distributions under varying loading conditions. Five different UGI designs were printed, including non-gradient, 1D in-plane, 2D in-plane, 1D out-of-plane, and 3D gradients (FIG. 27A, D, G; FIG. 6-7). Gradient mixing was visualized using fluorescent imaging by incorporating red and green fluorescent dyes into the two PUD inks. The white line in FIG. 27G indicates the position of the lower graph showing the out-of-plane (or z-direction) distribution of the two polyurethanes. In FIG. 27D, G, the brighter coloration along the diagonal position is attributed to the designed concentric infill printing paths, which result in slightly increased ink deposition in that area. However, this minor variation in ink deposition has negligible impact on UGI performance. Through in situ modulation of the material composition, these designs lead to different site-specific elastic moduli distributions (FIG. 27J, K; FIG. 7). Since the X-direction gradient design is identical for both the 1D and 2D in-plane gradient samples, their composition and modulus distributions along the X direction are the same. The top surface maximum principal Lagrange strain maps obtained from DIC for samples under 100% uniaxial global stretch show significant variations in strain distribution among different designs, particularly near the stiff regions and stiff/soft interfaces (FIG. 27B, E, H; FIG. 7-8). Here, a 100% uniaxial stretch ratio indicates the material is stretched to two times of its original length

$$( \frac{L - {L}_{0}}{{L}_{0}} = 100 \% ,$$

where L is the length after stretch and L₀ is the length before stretch). Finite Element Analysis (FEA) models were also created, presenting strain distributions consistent with experiments. These models provide valuable insights into stress/strain distribution under varying loading conditions (FIGS. 2-3, and 9). The non-gradient design exhibits stress and strain concentration at the soft-stiff interfaces (FIG. 2-3, 7-9). Due to the sharp modulus transition, significant strain mismatch occurs at the interface during loading, consequently resulting in elevated stress level. In this way, a failure is likely to initiate early, occurring at ~48% uniaxial strain (FIG. 7-8). Conversely, the UGI designs effectively reduce local stress and strain concentrations arising from the sharp contrast of elastic moduli between the stiff and soft components. Under 100% uniaxial tension, maximum principal Lagrange strains remain as low as 1.4%, 1.2%, and 1.1% for 1D in-plane, 2D in-plane, and 3D gradients in the stiff areas, respectively (FIG. 271). The strain progressively increases along the direction of decreasing modulus (FIG. 27B, E, H; FIG. 8), as softer materials undergo greater deformation under the identical external loads. Notably, the smooth strain transitions in 2D in-plane, and 3D UGIs significantly mitigate stress concentration (FIG. 2-3, 9). FIG. 8 shows excellent strain isolation for the 3D UGI, where the strain in the stiff region is as small as 2.7% while the uniaxial stretch ratio approaches 180%.

Furthermore, the initial failure points differ across designs. Non-gradient design tends to fail prematurely at the stiff-soft interface due to high stress and strain concentrations caused by the sharp modulus changes (FIG. 7-8). In contrast, UGIs exhibit failure within the soft region when the strain exceeds the intrinsic limit of the soft polyurethane (FIG. 27C, F, I; FIG. 9). These gradient designs effectively distribute stress and strain across the stiff-soft transition region, thereby significantly enhancing global failure resistance. FIG. 27M and FIG. 11 display force-displacement curves for different designs. The above results underscore the crucial role of stress distribution in crack initiation and illustrate the significance of gradient design that facilitates more uniform stress distribution.

### Versatility of UGI to Enable Strain-Insensitive Stretchable Materials and Devices

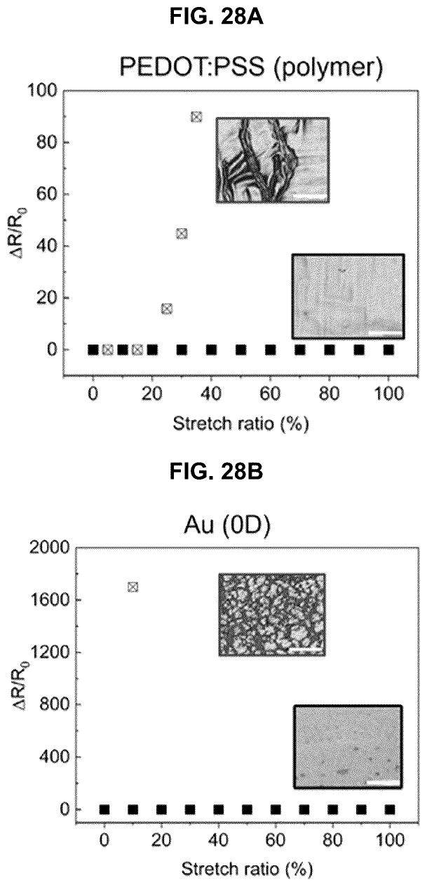

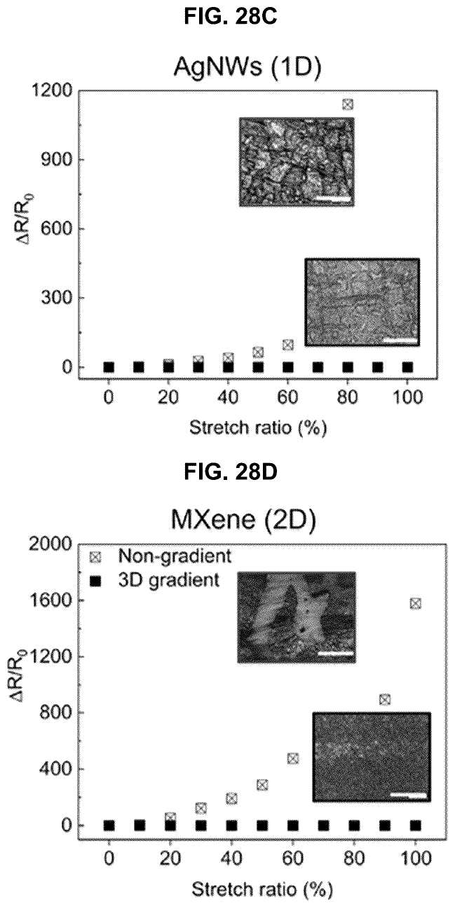

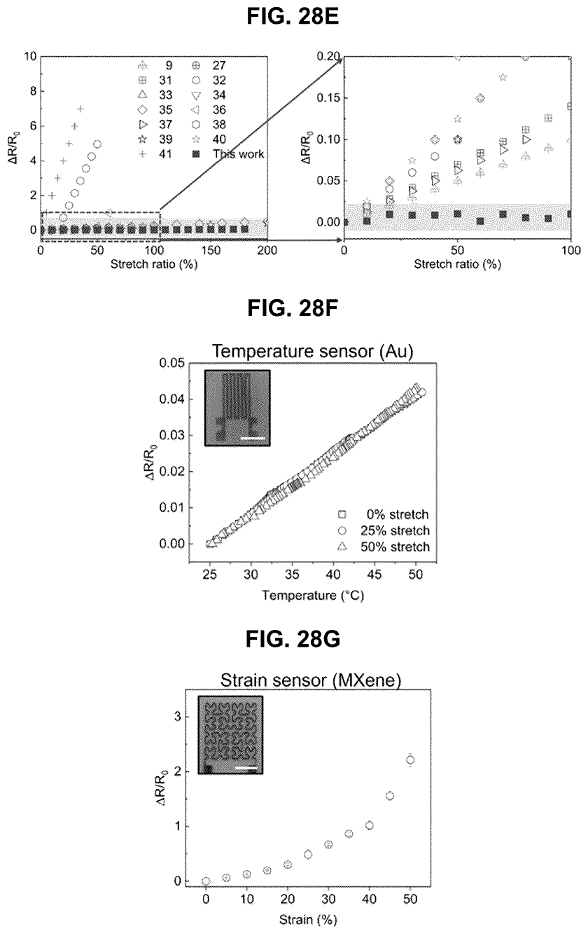

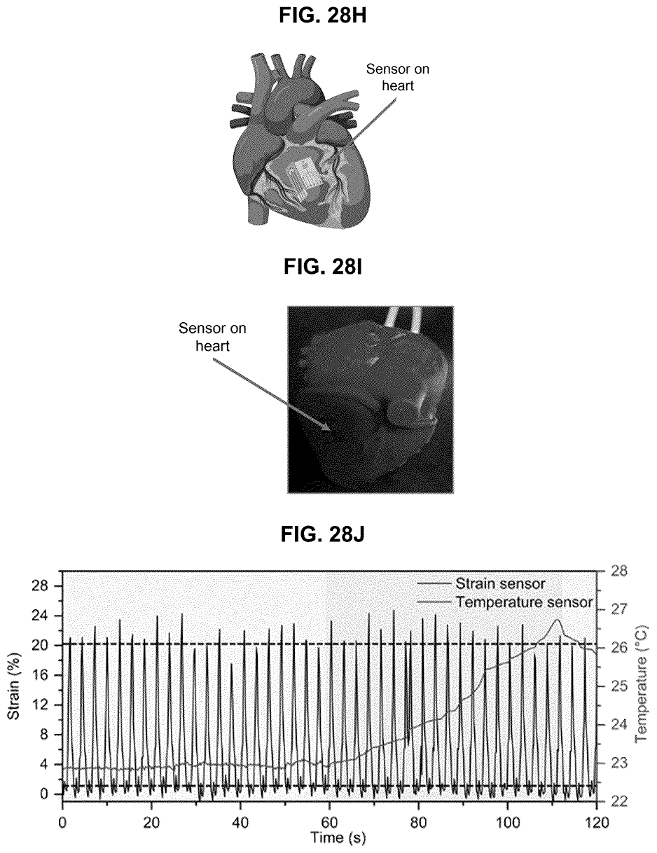

To demonstrate the versatility and universal applications of the UGI, conductive polymer films of PEDOT: PSS, 0D gold nanoparticle, 1D silver nanowires (AgNWs), and 2D MXene were printed on the 3D UGI and subjected them up to 100% uniaxial stretch ratio (see Tables 1 and 6 for detailed fabrication strategies). The printed Au film was sintered using a photonic flash sintering process detailed in FIG. 12. The conductive films printed on homogeneous soft PU substrates were also tested for comparison. As shown in FIG. 28A-D, all the conductive films on UGI show no visible cracks at a 100% stretch ratio, with the relative resistance change (RRC) (ΔR/R₀)<4.2%, whereas those on homogeneous substrates showing significant RRC on increases and microcracks leading to electrical failure. To validate the UGI's mechanical repeatability and stability, a 1,000-cycle tensile test was performed at a 50% uniaxial stretch ratio on a 3D gradient UGI. The results, detailed in FIG. 14, show a low RRC of less than 1.6%, demonstrating the 3D UGI's durability and resilience under cyclic loading. These results highlight the superior strain isolation properties of the UGI design compared to state-of-the-art stretchable electronics (FIG. 28E).

Next, the printing of electronic devices and sensors on the UGI was explored. A stretchable Au-based resistive temperature sensor is printed on the 3D UGI (FIG. 15), demonstrating a sensitivity of 1.6×10⁻³ °C⁻¹ across a 25 to 50 °C range, with stable sensitivity during stretching (FIG. 28F). Tools for cardiac physiological mapping are indispensable for the clinical identification and mechanistic understanding of excitation-contraction coupling, metabolic dysfunction, arrhythmia, and other conditions. Employing an artificial heart model, the real-time performance of the sensors was evaluated under dynamic conditions. To monitor the heart beating during diastole and systole, an MXene-based strain sensor was printed on the homogeneous soft substrate (FIG. 28G). The strain and temperature sensors were then affixed to an artificial heart (FIG. 28H-I), which pulsates every 2 s. The measurements showed that the strain sensor can catch the periodic diastole and systole process (FIG. 28J). Concurrently, the temperature sensor records temperature changes over time, maintaining high stability under repeated strain cycles (<1.15% resistance change during consistent temperature conditions). These results indicate that the UGI provides a robust platform to monitor real-time signals in a dynamic environment.

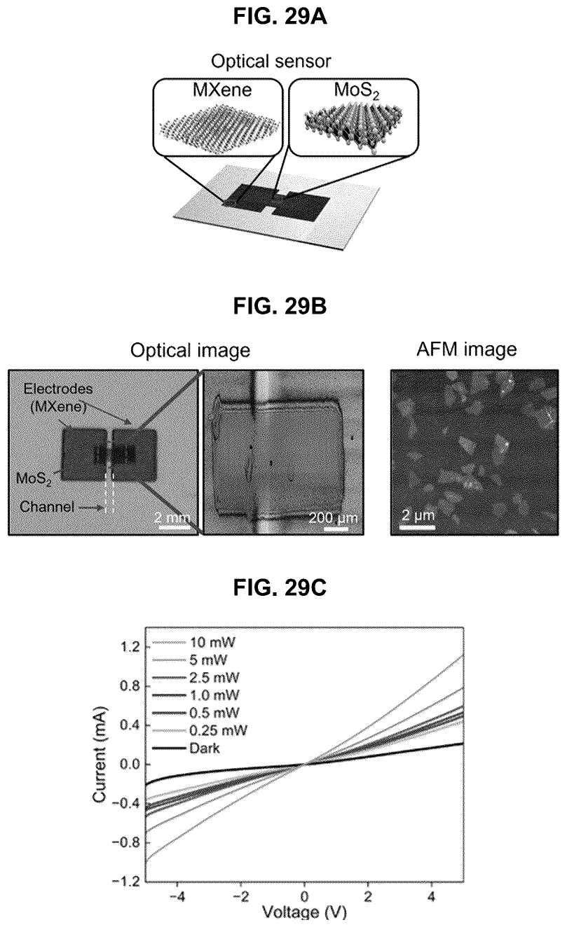

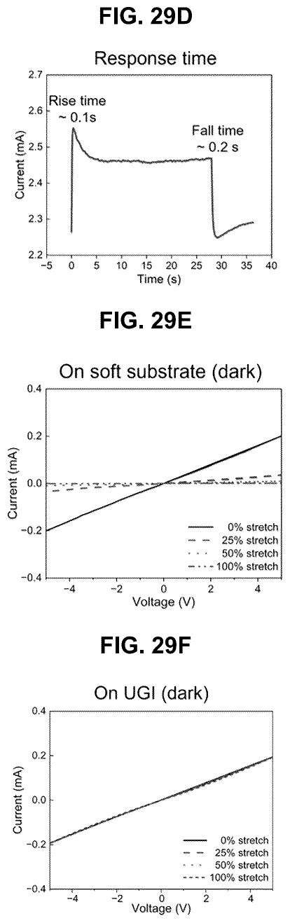

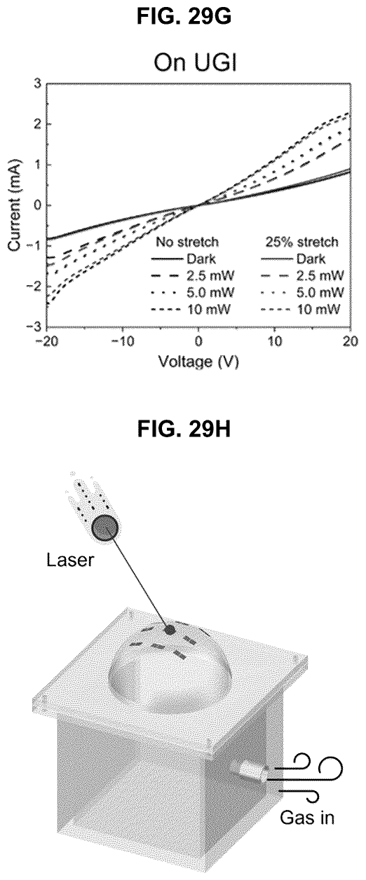

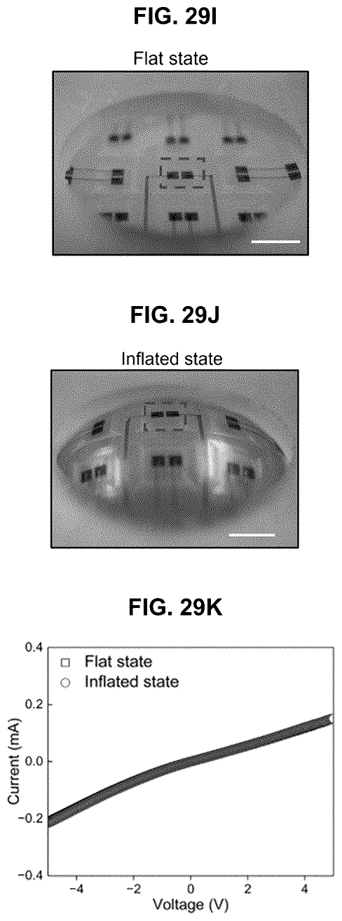

In addition to the stretchable resistance-based sensor, the UGI was employed to develop more sophisticated stretchable optoelectronic devices utilizing printed semiconducting 2D MoS₂, which transitions from an indirect to a direct bandgap in a monolayer regime and thus is ideal for optoelectronic applications in the atomically thin limit. As illustrated in FIG. 29A-B, the MoS₂ inks were printed as the channel material between the two printed MXene electrodes (FIG. 18), exhibiting a power-law relationship (R∝P, γ=−0.06), resulting in higher responsivities (R) at lower powers (P) (FIG. 29C; FIG. 19). At low laser intensity (0.15 W cm⁻²), the responsivity for the devices reached 0.62 A W⁻¹, which is among the highest of previously reported all-printed photodetectors in the visible light range. Further details on the responsivity calculation are provided in FIG. 19. Photodetector response times, with rise and fall constants of ~0.1 and 0.2 s, respectively, reflect the rapid generation and slower recombination of charge carriers facilitated by deep trap states (FIG. 29FD). A trade-off between response time and MoS₂ layer thickness is observed (FIG. 20). The all-printed photodetectors exhibited relatively fast response times compared to some literature using CVD-grown and mechanically exfoliated MoS₂ photodetectors (FIG. 20). The slower response times of CVD-grown and mechanically MoS₂ photodetectors, linked to interface traps at the MoS₂-SiO₂ interface, contrast with faster times on polyurethane substrate due to reduced trapping.

To assess the performance of photodetectors on both 3D gradient and soft interfaces, the dark current was recorded under various stretch ratios (FIG. 29E-F). Even under 100% uniaxial stretch, the dark current shows <2.9% change on the 3D gradient interface, whereas over 16700% decrease of dark current occurs on the soft substrate. Performance on 3D gradient interfaces under various laser powers and strains demonstrated <2.6% signal drift at 25% stretch (FIG. 29G), highlighting the robust strain isolation of the UGI design, crucial for applications in dynamically changing environments like tunable electronic eye cameras.

An array of nine photodetectors was printed and mounted on an inflatable hemispherical structure subjected to variable 2D strains to demonstrate advanced 3D sensing systems. (FIG. 29H-J; FIG. 21). The photodetector array exhibits remarkable strain isolation capabilities and <1.9% change of dark current under maximum in-plane strains of 57% in each direction (FIG. 29K). Finite element simulations (FIG. 22) revealed a smooth distribution of maximum principal strain and stress in response to the gradient modulus, mitigating the stress and strain mismatch at the interface between the photodetector and the soft substrate. Notably, the strain in the photodetectors on the UGI remains minimal, indicating the robustness of this design.

The 3D stretchable photodetector array can simultaneously detect both the light intensity and the position of incident light. When laser light (405 nm wavelength) directly strikes the center of the photodetector array, the central detector exhibits a dominant photocurrent of 139.6 μA, with the surrounding eight detectors showing negligible photocurrents (FIG. 29L). However, for oblique incident light to the right sensor, the most intense response emerges in the laser-light-focused photosensor (right sensor) (FIG. 29M; FIG. 23). These findings pave the way for the integration of stretchable photodetectors in versatile 3D sensing applications. Envisioning future advancements, combining these sensors with 3D-printed laser, visible, and infrared detectors could revolutionize bionic eye development, offering comprehensive all-angle and all-wavelength visual capabilities.

### All Printed Strain-Insensitive On-Skin Sensors for Continuous Health Monitoring

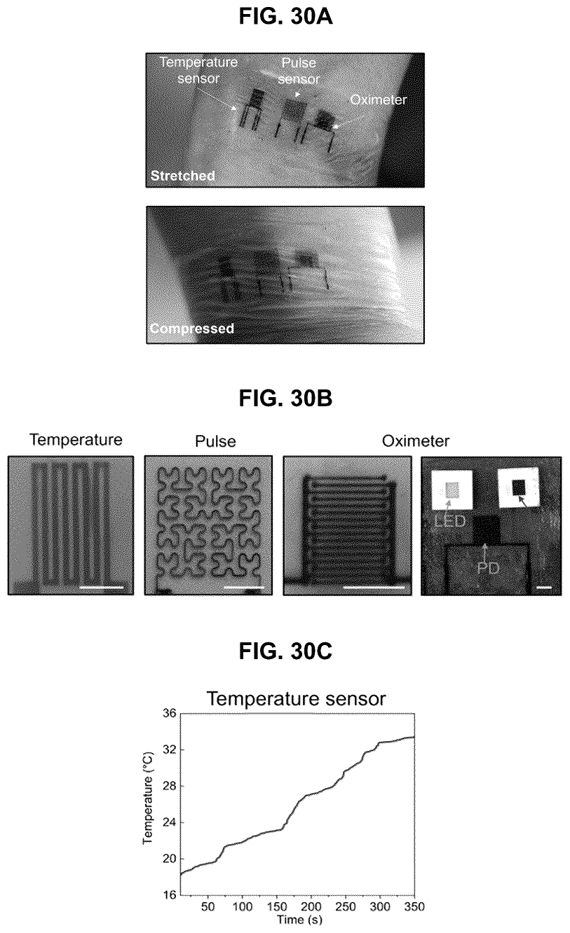

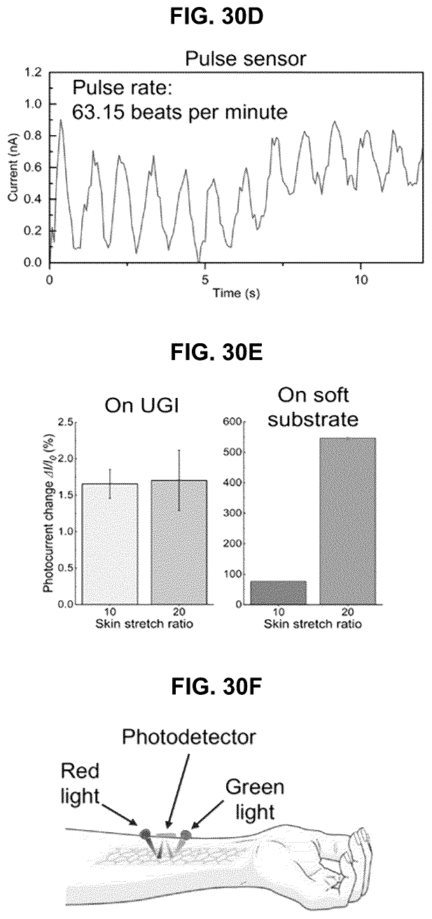

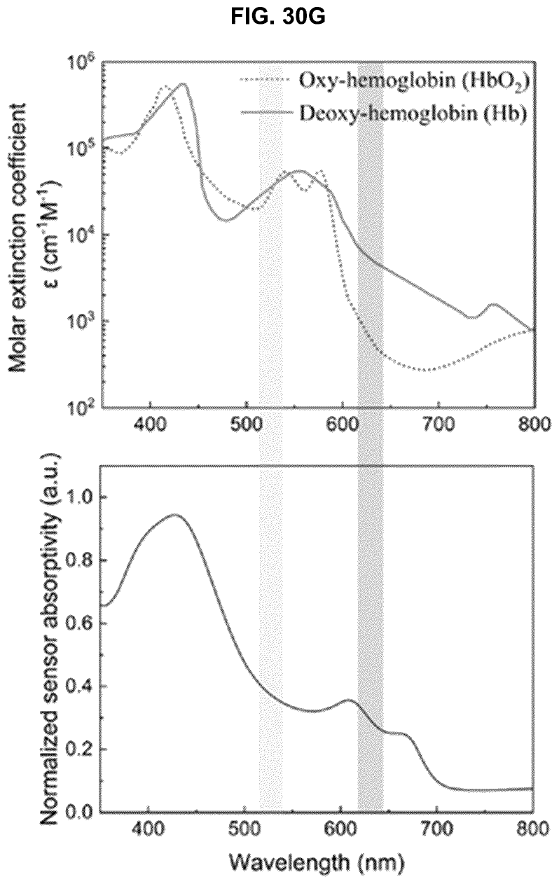

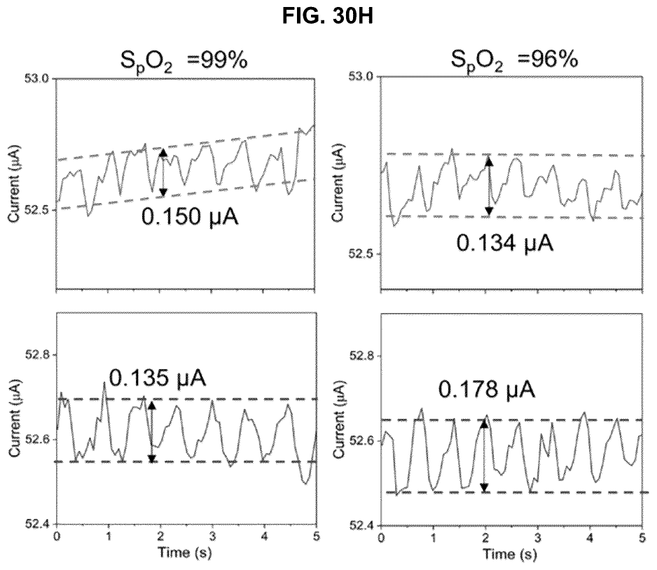

The UGI serves as a versatile platform for printing multimodal sensors for on-skin health monitoring. Here, the harmonious integration of temperature and blood oxygen saturation sensors was demonstrated on the stiff region of the UGI and a pulse sensor on the soft region to detect pulse-induced strain changes, creating a stretchable system integrated seamlessly with the skin (FIG. 30A-B). To ensure robust and reliable measurements while maintaining user comfort, the connection setup for the skin-mounted sensors is illustrated in FIG. 24, providing a clear depiction of how the measurements were conducted.

The temperature sensor accurately tracks skin temperature fluctuations, capturing changes from 18.2 to 33.4 °C as the participant moves from a cold to a warm environment (FIG. 30C). The pulse sensor using printed MXene patterns dynamically records the electrical current variations that correspond with the heartbeat, enabling precise heart rate monitoring by counting peaks over a minute (FIG. 30D). Pulse oximetry, a widely utilized non-invasive method, evaluates tissue oxygen saturation by optically determining the relative concentrations of oxyhemoglobin (HbO₂) and deoxyhemoglobin (Hb) in blood using photoplethysmography (PPG). The MoS₂-based photodetector is integrated with green and red LED lights (FIG. 30F) to create a stretchable pulse oximeter that conforms to the skin. In this setup, reflected light is modulated by the dynamic volumes of arterial blood, affecting light absorption detected by the photodetector. This modulation, correlating with the heart's systolic and diastolic phases, produces a pulsatile optical signal that can be used to determine the blood oxygenation levels (FIG. 25). HbO₂ and Hb exhibit different absorption rates of oxygenated and deoxygenated hemoglobin at red and green wavelengths (FIG. 30G). Blood oxygen saturation (SpO₂) can be calculated by determining the light absorption ratio at these two wavelengths (FIG. 25). FIG. 30H shows the corresponding signals when SpO₂ decreases from 99% to 96% during a breath hold test.

Motion artifacts pose significant challenges in wearable electronics for health monitoring, leading to potential misinterpretations and severe health implications. To demonstrate the superior immunity to strain/motion artifacts of printed UGI, photodetectors were printed on both UGI and soft substrates, respectively, and then tested on skin with induced strain deformations while recording the signals (FIG. 26). The oximeter on the UGI showed neglectable signal deviation (<1.7% change of measured photocurrent under ~20% uniaxial strain), as depicted in FIG. 30E, demonstrating by far the best performances in reducing motion artifacts among previously reported wearable devices (Tables 4-6). In contrast, the oximeter on the soft substrate experienced over >547% signal variance (FIG. 30E), underscoring the UGI's superior capability for stable biological signal capture amidst skin deformations.

TABLE 4
Typical Stretchable Materials and their Performance

| | Plot number | | |
|---|---|---|---|
| | in main | | |
| Ref | content | Resistance increase | Methods |
| [1] | 9 | The resistance of the elastic conductors | Apply pre-strain |
| | | remained the same under high bending | |
| | | strain and increased slightly (≈14%) at a | |
| | | tensile strain of 100% | |
| [2] | 27 | Larger pre-strain (20, 30, 40%) resulted in | Apply pre-strain |
| | | stable conductance | |
| [3] | 31 | Stable of stretching at ε = 0.7 | Intrinsically stretchable |
| | | | material |
| [4] | 32 | Resistance maintained at 60% (longitudinal), | Intrinsically stretchable |
| | | 140% (transverse) strain | material |
| [5] | 33 | The resistance keeps increasing slightly | Intrinsically stretchable |
| | | | material |
| [6] | 34 | Conductivity maintained up to 50% strain | Wrinkle and Serpentine |
| [7] | 35 | ΔR/R0 = 0.1 at 80% strain | Wrinkle |
| [8] | 36 | Conductivity maintained up to 50% strain | Serpentine |
| [9] | 37 | Resistance slightly increased up to 200% | Serpentine |
| | | strain | |
| [10] | 38 | Conductivity maintained up to 100% strain | Serpentine |
| [11] | 39 | Resistance slightly increased up to 200% | Rigid island |
| | | strain | |
| [12] | 40 | The resistance keeps increasing slightly | Gradient substrate |
| [13] | 41 | The resistance keeps increasing slightly | Rigid island |

[1] Kim et al., Adv. Mater. 23(25): 2865-2869 (2011)
[2] Miyamoto et al., Nat. Nanotechnol. 12(9): 907-913 (2017).
[3] Shin et al., Adv. Mater., 26 (22): 3706-3711 (2014).
[4] Moon et al., Adv. Mater. 25 (19): 2707-2712 (2013).
[5] Lee et al., Adv. Mater. 24(25): 3326-3332 (2012)
[6] Hyun et al., Adv. Mater. 23(26): 2946-2950 (2011).
[7] Feig et al., Nat. Commun. 9(1): 2740 (2018).
[8] Park et al., Sci. Transl. Med. 8(344): 344ra86 (2016).
[9] Ma et al., ACS Nano 9(11): 10876-10886 (2015).
[10] Choi et al., ACS Nano 9(6): 6626-6633 (2015).
[11] Libanori et al., Nat. Commun. 3: 1265 (2012).
[12] Yang et al., 2D Mater. 4(3): 035220 (2017).
[13] Zhao et al., Science 378(6625): 1222-1227 (2022).

TABLE 5
Literature Comparison for FIG. 21B

| | Plot | | | |
|---|---|---|---|---|
| | label | Printing | Response | Responsivity |
| Ref | number | method | time (ms) | (A/W) |
| | This work | AJP | 120 | 0.14 |
| | This work | AJP | 150 | 0.37 |
| | This work | AJP | 4930 | 0.62 |
| [14] | 1 | Inkjet | 60 | 3.6 × 10⁻⁵ |
| [15] | 2 | Inkjet | 5 | 0.05 |
| [16] | 3 | Inkjet | 1 | 0.001 |
| [17] | 4 | Electrohydrodynamic | 1000 | 0.004 |
| [18] | 5 | Laser printing | 200 | 0.0013 |
| [19] | 6 | Inkjet | 23 | 0.01 |
| [20] | 7 | Inkjet | 1700 | 0.002 |

[14] Li et al., Adv. Funct. Mater. 24: 6524-6531 (2014).
[15] Seo et al., ACS Appl. Mater. Interfaces 11(6): 5675-5681 (2019).
[16] McManus et al., Nat. Nanotechnol. 12(4): 343-350 (2017).
[17] Alzakia et al., ACS Appl. Mater. Interfaces 12(25): 28840-28851 (2020).
[18] Mazaheri et al., Nanoscale 12(37): 19068-19074 (2020).
[19] Kong et al., Adv. Eng. Mater. 25(2): 22200946 1 (2023).
[20] Kim et al., ACS Nano 11(10): 10273-10280 (2017).

TABLE 6
Typical Motion Artifact-Free Device Performance

| | | Percent signal | Applied |
|---|---|---|---|
| Ref | Sensor type | variance/drift | strain |
| This work | Oximeter | <1.7% | ≈20% |
| [21] | Hydration sensor | ≈14.7% | ≈10% |
| [22] | Epidermal antenna | ≈80% | ≈20% |
| [23] | EMG electrode array | ≈60% | Unknown |
| [24] | Solid-state analyte sensor | ≈21% | Unknown |
| [25] | Pulse sensor | ≈50% | Unknown |

[21] Ershad et al., Nat. Commun. 11(1): 3823 (2020).
[22] Wang et al., Biosens. Bioelectron. 253: 116150 (2024).
[23] Jiang et al., Nature 614(7948): 456-462 (2023).
[24] Arwani et al., Nat. Mater. 23(8): 1115-1122 (2024).
[24] Jeong et al., Adv. Funct. Mater. 32: 2206461 (2022).

In summary, the printed universal gradient interfaces (UGIs) with submicron resolution and 3D modulus distributions disclosed herein provide unprecedented strain isolation and a versatile platform for realizing strain-insensitive stretchable devices with over 180% uniaxial stretchability. The versatility of the aerosol-based multi-materials printing (AMMP) method allows for integrating a diverse range of structural and functional materials into stretchable systems with precise control over global device structures and local properties with unparalleled spatial resolution. This UGI platform enables multifunctional stretchable devices for on-skin sensing of temperature, light, and blood oxygen, which allows truly motion artifact-free health monitoring. The AMMP has the potential for high throughput printing by deploying a large array of printing nozzles to scale up the printing processes. This innovation paves the way for advanced wearable and implantable devices for real-time health monitoring and therapeutics, as well as for soft and hybrid robotics with integrated soft-stiff gradient interfaces that are essential in natural musculoskeletal structures.

1. A printed universal gradient interface for stretchable and strain-insensitive bioelectronics, the universal gradient interface comprising a multi-polymer gradient extending between a first polymer end and a second polymer end, the first polymer end comprising a first polymer content, the second polymer end comprising a second polymer content, and the multi-polymer gradient comprising a graded weight ratio of the first polymer content to the second polymer content of between 1:0 and 0:1 from the first polymer end to the second polymer end.

2. The universal gradient interface of claim 1, wherein each of the first polymer content and the second polymer content comprises a polyurethane polymer, a polyurethane dispersion (PUD) polymer, a thermoplastic polyurethane (TPU) polymer, a polynitrile polymer, a polyether polymer, a polycarbonate polymer, a polyalcohol polymer, a polyethylene polymer, a polyethylene oxide polymer, a polyethylene glycol polymer, a polypropylene polymer, a polyacrylate polymer, a polyester polymer, a polyvinyl polymer, a polyphosphazene polymer, a polysulfone polymer, a polysiloxane polymer, a polyamide polymer, a polylactic acid polymer, a polyacrylate polymer, a polyimide polymer, a fluoropolymer, or combinations thereof.

3. The universal gradient interface of claim 1, wherein the multi-polymer gradient comprises a polyurethane dispersion (PUD) gradient extending between a first PUD end and a second PUD end, the first PUD end comprising a first PUD content, the second PUD end comprising a second PUD content, and the multi-polymer gradient comprising a graded weight ratio of the first PUD content to the second PUD content of between 1:0 and 0:1 from the first PUD end to the second PUD end.

4. The universal gradient interface of claim 3, wherein each of the first PUD content and the second PUD content comprises one or more of a polyether-based PUD, a polyester-based PUD, or a polycarbonate-based PUD.

5. The universal gradient interface of claim 3, wherein the first PUD content comprises flexible polyester polyol segments and the second PUD content comprises stiff polyester polyol segments derived from methylene diphenyl diisocyanate (MDI).

6. The universal gradient interface of claim 1, wherein the first polymer content has a Young's modulus of about 0.05 MPa to about 80 MPa and the second polymer content has a Young's modulus of about 100 MPa to about 50 GPa.

7. The universal gradient interface of claim 1, wherein the multi-polymer gradient has a Young's modulus ranging from about 0.05 MPa at the first polymer end to about 50 GPa at the second polymer end.

8. The universal gradient interface of claim 1, wherein the universal gradient interface has a thickness of about 0.5 μm to about 2 mm.

9. The universal gradient interface of claim 1, wherein the second polymer end is configured to functionally connect to an electrically conductive material.

10. The universal gradient interface of claim 9, wherein the electrically conductive material comprises one or more of a metal, a semiconductor, a conductive polymer, or an electrode.

11. A printed bioelectronic device, comprising:
a universal gradient interface comprising a multi-polymer gradient extending between a first polymer end and a second polymer end, the first polymer end comprising a first polymer content, the second polymer end comprising a second polymer content, and the multi-polymer gradient comprising a graded weight ratio of the first polymer content to the second polymer content of between 1:0 and 0:1 from the first polymer end to the second polymer end; and
an electrically conductive material functionally connected to the second polymer end of the universal gradient interface.

12. The bioelectronic device of claim 11, wherein each of the first polymer content and the second polymer content comprises a polyurethane polymer, a polyurethane dispersion (PUD) polymer, a thermoplastic polyurethane (TPU) polymer, a polynitrile polymer, a polyether polymer, a polycarbonate polymer, a polyalcohol polymer, a polyethylene polymer, a polyethylene oxide polymer, a polyethylene glycol polymer, a polypropylene polymer, a polyacrylate polymer, a polyester polymer, a polyvinyl polymer, a polyphosphazene polymer, a polysulfone polymer, a polysiloxane polymer, a polyamide polymer, a polylactic acid polymer, a polyacrylate polymer, a polyimide polymer, a fluoropolymer, or combinations thereof.

13. The bioelectronic device of claim 11, wherein the multi-polymer gradient of the universal gradient interface comprises a polyurethane dispersion (PUD) gradient extending between a first PUD end and a second PUD end, the first PUD end comprising a first PUD content, the second PUD end comprising a second PUD content, and the multi-polymer gradient comprising a graded weight ratio of the first PUD content to the second PUD content of between 1:0 and 0:1 from the first PUD end to the second PUD end.

14. The bioelectronic device of claim 11, wherein the electrically conductive material comprises one or more of a metal, a semiconductor, a conductive polymer, or an electrode.

15. The bioelectronic device of claim 11, wherein the first polymer end of the universal gradient interface is configured to functionally connect to a biological surface.

16. The bioelectronic device of claim 15, wherein the biological surface comprises a mammalian tissue or organ.

17. (canceled)

18. The bioelectronic device of claim 11, wherein the bioelectronic device is a wearable bioelectronic device.

19-22. (canceled)

23. A method of making a universal gradient interface for stretchable and strain-insensitive bioelectronics, the method comprising:
mixing a first aerosolized ink stream comprising a first polymer and a second aerosolized ink stream comprising a second polymer using a carrier gas to form an aerosolized polymer mixture; and
aerosol jet printing the aerosolized polymer mixture onto a substrate material using a sheath gas such that the first aerosolized ink stream comprising the first polymer is first deposited onto the substrate material to form a first polymer end comprising the first polymer;
wherein flow rates of the first aerosolized ink stream and the second aerosolized ink stream are continuously adjusted to form a multi-polymer gradient extending between the first polymer end and a second polymer end comprising the second polymer, the multi-polymer gradient comprising a graded weight ratio of the first polymer to the second polymer of between 1:0 and 0:1 from the first polymer end to the second polymer end.

24. The method of claim 23, wherein the substrate material is treated with plasma to improve surface hydrophilicity prior to aerosol jet printing.

25. (canceled)

26. The method of claim 23, further comprising aerosol jet printing a third aerosolized ink stream comprising an electrically conductive material onto the second polymer end, thereby functionally connecting the second polymer end to the electrically conductive material.

27-32. (canceled)
